(19) United States
(12) Patent Application Publication
(10) Pub. No.: US 2026/0117308 A1
(43) Pub. Date: Apr. 30, 2026

(54) SYSTEMS AND METHODS FOR MULTI-ANALYTE DETECTION OF CANCER

(71) Applicant: Predicine, Inc., Hayward, CA (US)

(72) Inventors: Pan DU, Dublin, CA (US); Binggang XIANG, Pleasanton, CA (US); Chao DAI, Newark, CA (US); Shujun LUO, Castro Valley, CA (US); Shidong JIA, Palo Alto, CA (US)

(21) Appl. No.: 19/169,558

(22) Filed: Apr. 3, 2025

Related U.S. Application Data
(63) Continuation of application No. PCT/US2023/075980, filed on Oct. 4, 2023.
(60) Provisional application No. 63/413,433, filed on Oct. 5, 2022.
(60) Provisional application No. 63/445,201, filed on Feb. 13, 2023.
(60) Provisional application No. 63/445,145, filed on Feb. 13, 2023.
(60) Provisional application No. 63/445,150, filed on Feb. 13, 2023.

(51) Int. Cl. C12Q 1/6886; G16B 20/10; G16B 20/20

(52) U.S. Cl. CPC C12Q 1/6886; G16B 20/10; G16B 20/20; C12Q 2600/154; C12Q 2600/156

(57) ABSTRACT

Provided herein are methods and systems for detection of cancer. The method may comprise using nucleic acids from a urine sample. The methods may comprise assaying nucleic acids in urine to detect a set of biomarkers from samples. The methods may comprise processing the set of biomarkers to determine the presence of a cancer or cancer parameters. The processing may be performed by an algorithm.

### CROSS-REFERENCE

This application is a continuation of International Application No. PCT/US2023/075980, filed Oct. 4, 2023, which claims the benefit of U.S. Application No. 63/413,433, filed Oct. 5, 2022, U.S. Application No. 63/445,201, filed Feb. 13, 2023, U.S. Application No. 63/445,145, filed Feb. 13, 2023, and U.S. Application No. 63/445,150, filed Feb. 13, 2023, each of which is incorporated by reference herein in its entirety.

### BACKGROUND

Cancer is a leading cause of deaths worldwide. Detection of cancer in individuals may be critical for providing treatment and improving patient outcomes. Cancer may be caused by genetic aberration which may lead to unregulated growth of calls. Detection of the genetic aberrations may be important for the detection of cancer. Sequencing of nucleic acids in a sample from a patient may be used to detect genetic aberrations.

### SUMMARY

Provided herein are systems and methods for detection of the presence or absence of cancer in a subject. The systems and methods provided herein comprises assaying polynucleotides to identify biomarkers of cancers in a subject. Detection of a type of cancer or the specific biomarkers for a given cancer may allow an effective treatment to be provided to an individual and may result in improved outcomes. For multiple types of cancer, the particular biomarkers that indicate a particular cancer type (or subtype) may be used to identify a prognosis for an individual suffering from the cancer. In order to provide accurate detection and prognosis for a cancer, multiple analytes may be examined. By analyzing an increased number of analytes (and sets of biomarkers from the analytes), the detection of a cancer (or cancer parameter) may be improved and may allow for the recommendation of an effective treatment, and may also allow for the prognosis to be more accurate.

In an aspect, the present disclosure provides a method for detecting a presence or an absence of cancer in a subject, comprising (a) assaying cell-free deoxyribonucleic acid (cfDNA) molecules from a biological sample obtained or derived from the subject, wherein the biological sample comprises a urine sample; (b) detecting a set of biomarkers from the cfDNA molecules wherein the set of biomarkers comprise differentially expressed markers or variants; (c) computer processing the set of biomarkers to detect the presence or the absence of the cancer in the subject. The method of claim 1, wherein the biological sample is obtained or derived from the subject using an ethylenediaminetetraacetic acid (EDTA) collection tube, a cell-free RNA collection tube, or a cell-free deoxyribonucleic acid (DNA) collection tube, other blood collection tube, and CTC collection tubes.

In some embodiments, (a) comprises subjecting the biological sample to conditions that are sufficient to isolate, enrich, or extract the cfDNA molecules. In some embodiments, at least one of the cfDNA molecules are assayed using nucleic acid sequencing to produce nucleic acid sequencing reads. In some embodiments, the method further comprises filtering at least a subset of the nucleic acid sequencing reads based on a quality score. In some embodiments, the method further comprises performing error correction on the nucleic acid sequencing reads using sample barcodes or molecular barcodes attached to at least one of the cfDNA molecules. In some embodiments, the method further comprises performing at least one of single-stranded consensus calling and double-stranded consensus calling on the nucleic acid sequencing reads, thereby suppressing sequencing and PCR errors in the nucleic acid sequencing reads.

In some embodiments, cfDNA molecules are assayed using DNA sequencing. In some embodiments, the DNA sequencing is selected from the group consisting of: next-generation sequencing, whole genome sequencing, low-pass sequencing, targeted sequencing, whole exome sequencing, methylation-aware sequencing, bisulfite sequencing, and a combination thereof. In some embodiments, the DNA sequencing comprises targeted sequencing. In some embodiments, the nucleic acid sequencing comprises nucleic acid amplification. In some embodiments, the nucleic acid amplification comprises polymerase chain reaction (PCR) or isothermal amplification. In some embodiments, the least one of the cfDNA molecules are assayed using a polymerase chain reaction (PCR) assay, microarray, or an isothermal amplification.

In some embodiments, the cancer is selected from the group consisting of: breast cancer, lung cancer, prostate cancer, colorectal cancer, melanoma, bladder cancer, non-Hodgkin lymphoma, kidney cancer, endometrial cancer, leukemia, pancreatic cancer, thyroid cancer, and liver cancer, and any combination thereof. In some embodiments, the cancer comprises bladder cancer. In some embodiments, the cancer comprises kidney cancer. In some embodiments, the bladder cancer comprise non-muscle invasive bladder cancer. In some embodiments, the subject is asymptomatic for the cancer.

In some embodiments, (b) comprises detecting the presence or the absence of the cancer in the subject at an accuracy of at least about 60%, at least about 70%, at least about 75%, at least about 80%, at least about 85%, at least about 90%, at least about 95%, at least about 98%, or at least about 99%. In some embodiments, (b) comprises detecting the presence or the absence of the cancer in the subject at a sensitivity of at least about 60%, at least about 70%, at least about 75%, at least about 80%, at least about 85%, at least about 90%, at least about 95%, at least about 98%, or at least about 99%. In some embodiments, (b) comprises detecting the presence or the absence of the cancer in the subject at a specificity of at least about 60%, at least about 70%, at least about 75%, at least about 80%, at least about 85%, at least about 90%, at least about 95%, at least about 98%, or at least about 99%. In some embodiments, (b) comprises detecting the presence or the absence of the cancer in the subject at a positive predictive value of at least about 60%, at least about 70%, at least about 75%, at least about 80%, at least about 85%, at least about 90%, at least about 95%, at least about 98%, or at least about 99%. In some embodiments, (b) comprises detecting the presence or the absence of the cancer in the subject at a negative predictive value of at least about 60%, at least about 70%, at least about 75%, at least about 80%, at least about 85%, at least about 90%, at least about 95%, at least about 98%, or at least about 99%.

In some embodiments, the biological sample is obtained or derived from the subject prior to the subject receiving a therapy for the cancer. In some embodiments, the biological sample is obtained or derived from the subject during a therapy for the cancer. In some embodiments, the biological sample is obtained or derived from the subject after receiving a therapy for the cancer. In some embodiments, the therapy is selected from the group consisting of: surgical resection, chemotherapy, radiotherapy, immunotherapy, cell therapy, adjuvant therapy, neoadjuvant therapy, androgen deprivation therapy, and a combination thereof. In some embodiments, the method further comprises identifying a clinical intervention for the subject based at least in part on the detected presence or the absence of the cancer. In some embodiments, the clinical intervention is selected from a plurality of clinical interventions. In some embodiments, the clinical intervention is selected from the group consisting of: surgical resection, chemotherapy, radiotherapy, immunotherapy, adjuvant therapy, neoadjuvant therapy, androgen deprivation therapy, and a combination thereof. In some embodiments, the method further comprises administering the clinical intervention to the subject.

In some embodiments, the set of biomarkers comprises quantitative measures of a set of cancer-associated genomic loci. In some embodiments, the set of cancer-associated genomic loci comprises one or more members selected from the group consisting of genes listed in Table 1. In some embodiments, the set of cancer-associated genomic loci comprises 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 1. In some embodiments, the set of cancer-associated genomic loci comprises PTEN, TP53 or RB1. In some embodiments, the set of cancer-associated genomic loci comprises PTEN. In some embodiments, the set of cancer-associated genomic loci comprises FGFR3 or ERBB2. In some embodiments, the set of cancer-associated genomic loci comprises one or more members selected from the group consisting of genes listed in Table 2. In some embodiments, the set of cancer-associated genomic loci comprises 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 2.

In some embodiments, the method further comprises using probes configured to selectively enrich the biological sample for nucleic acid molecules corresponding to a set of genomic loci. In some embodiments, the probes comprise nucleic acid primers. In some embodiments, the probes comprise nucleic acid capture probes. In some embodiments, the probes have sequence complementarity with at least a portion of nucleic acid sequences of the set of genomic loci. In some embodiments, the probes comprise at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 different probes.

In some embodiments, the method further comprises determining a likelihood of the determination of the presence or the absence of the cancer in the subject. In some embodiments, the method further comprises monitoring the presence or the absence of the cancer in the subject, wherein the monitoring comprises assessing the presence or the absence of the cancer in the subject at each of a plurality of time points. In some embodiments, a difference in the assessment of the presence or the absence of the cancer in the subject among the plurality of time points is indicative of one or more clinical indications selected from the group consisting of: (i) a diagnosis of the cancer, (ii) a prognosis of the cancer, and (iii) an efficacy or non-efficacy of a course of treatment for treating the cancer of the subject.

In some embodiments, the prognosis comprises an expected progression-free survival (PFS) or overall survival (OS). In some embodiments, the set of biomarkers from the cfDNA molecules comprise tumor-associated alterations selected from the group consisting of: copy number alterations (CNAs), copy number losses (CNLs), single nucleotide variants (SNVs), insertions or deletions (indels), and rearrangements. In some embodiments, the set of biomarkers from the cfDNA molecules comprise copy number variation. In some embodiments, the set of biomarkers from the cfDNA molecules comprise copy number losses. In some embodiments, the set of biomarkers from the cfDNA molecules comprise single nucleotide variants.

In some embodiments, the method further comprises determining, among the set of biomarkers, a mutant allele frequency of a set of somatic mutations. In some embodiments, the method further comprises determining a blood copy number burden based on copy number alterations or copy number losses of the set of biomarkers. In some embodiments, the method further comprises determining a circulating tumor DNA (ctDNA) fraction of the cancer of the subject based at least in part on the set of mutant allele frequencies. In some embodiments, the method further comprises determining a tumor mutational burden (TMB) of the cancer of the subject based at least in part on the set of mutant allele frequencies. In some embodiments, the method further comprises determining a plasma tumor mutational burden (TMB) of the cancer of the subject based at least in part on the set of mutant allele frequencies comprising microsatellites. In some embodiments, the method further comprises determining an abnormality score of the cancer of the subject based at least in part on the set of mutant allele frequencies.

In an aspect, the present disclosure provides a method for detecting a presence or an absence of cancer in a subject, comprising: (a) providing cell-free deoxyribonucleic acid (cfDNA) molecules from a biological sample obtained or derived from the subject, wherein the biological sample comprises a urine sample; (b) hybridizing the cfDNA molecules or derivatives thereof to a plurality of nucleic acid capture probes to generate a plurality of enriched cfDNA molecules; (c) sequencing nucleic acids of the plurality of enriched cfDNA molecules to generate sequencing data; (d) computer processing the sequencing data to detect a set of biomarkers; and (e) based at least on the presence of the set of biomarkers, detecting the presence or the absence of the cancer in the subject.

In an aspect, the present disclosure provides a method for detecting a presence or an absence of cancer in a subject, comprising: (a) providing cell-free deoxyribonucleic acid (cfDNA) molecules from a biological sample obtained or derived from the subject; (b) performing a first sequencing assay on the cfDNA, or derivatives thereof, to generate a copy number data of at least one region of a genome of a subject, wherein the sequencing is performed at a depth of no more than 10×; (c) performing a second sequencing assay on the cfDNA molecules or derivatives thereof, wherein the second sequencing assay comprises a whole exome sequencing assay or a methylation-aware sequencing assay to generate sequencing data; (d) computer processing the copy number data and the sequencing data to detect a set of biomarkers; and (e) based at least on the presence of the set of biomarkers, detecting the presence or the absence of the cancer in the subject.

In some embodiments, the biological sample is obtained or derived from the subject using an ethylenediaminetetraacetic acid (EDTA) collection tube, a cell-free RNA collection tube, or a cell-free deoxyribonucleic acid (DNA) collection tube, other blood collection tube, and CTC collection tubes. In some embodiments, (a) comprises subjecting the biological sample to conditions that are sufficient to isolate, enrich, or extract the cfDNA molecules.

In some embodiments, the biological sample comprises a urine, blood, or cerebrospinal sample. In some embodiments, the method further comprises filtering at least a subset of the nucleic acid sequencing reads based on a quality score. In some embodiments, the method further comprises performing error correction on the nucleic acid sequencing reads using sample barcodes or molecular barcodes attached to at least one of the cfDNA molecules. In some embodiments, the method further comprises performing at least one of single-stranded consensus calling and double-stranded consensus calling on the nucleic acid sequencing reads, thereby suppressing sequencing and PCR errors in the nucleic acid sequencing reads. In some embodiments, (b) or (c) comprises nucleic acid amplification. In some embodiments, the nucleic acid amplification comprises polymerase chain reaction (PCR) or isothermal amplification. In some embodiments, at least one of the cfDNA molecules are assayed using a polymerase chain reaction (PCR) assay, microarray, or an isothermal amplification. In some embodiments, the cancer is selected from the group consisting of: breast cancer, lung cancer, prostate cancer, colorectal cancer, melanoma, bladder cancer, non-Hodgkin lymphoma, kidney cancer, endometrial cancer, leukemia, pancreatic cancer, thyroid cancer, and liver cancer, and any combination thereof. In some embodiments, the cancer comprises bladder cancer. In some embodiments, the bladder cancer comprise non-muscle invasive bladder cancer. In some embodiments, the subject is asymptomatic for the cancer.

In some embodiments, (d) comprises detecting the presence or the absence of the cancer in the subject at an accuracy of at least about 60%, at least about 70%, at least about 75%, at least about 80%, at least about 85%, at least about 90%, at least about 95%, at least about 98%, or at least about 99%. In some embodiments, (d) comprises detecting the presence or the absence of the cancer in the subject at a sensitivity of at least about 60%, at least about 70%, at least about 75%, at least about 80%, at least about 85%, at least about 90%, at least about 95%, at least about 98%, or at least about 99%. In some embodiments, (d) comprises detecting the presence or the absence of the cancer in the subject at a specificity of at least about 60%, at least about 70%, at least about 75%, at least about 80%, at least about 85%, at least about 90%, at least about 95%, at least about 98%, or at least about 99%. In some embodiments, (d) comprises detecting the presence or the absence of the cancer in the subject at a positive predictive value of at least about 60%, at least about 70%, at least about 75%, at least about 80%, at least about 85%, at least about 90%, at least about 95%, at least about 98%, or at least about 99%. In some embodiments, (d) comprises detecting the presence or the absence of the cancer in the subject at a negative predictive value of at least about 60%, at least about 70%, at least about 75%, at least about 80%, at least about 85%, at least about 90%, at least about 95%, at least about 98%, or at least about 99%.

In some embodiments, the biological sample is obtained or derived from the subject prior to the subject receiving a therapy for the cancer. In some embodiments, the biological sample is obtained or derived from the subject during a therapy for the cancer. In some embodiments, the biological sample is obtained or derived from the subject after receiving a therapy for the cancer. In some embodiments, the therapy is selected from the group consisting of: surgical resection, chemotherapy, radiotherapy, immunotherapy, cell therapy, adjuvant therapy, neoadjuvant therapy, androgen deprivation therapy, and a combination thereof.

In some embodiments, the method further comprises identifying a clinical intervention for the subject based at least in part on the detected presence or the absence of the cancer. In some embodiments, the clinical intervention is selected from a plurality of clinical interventions. In some embodiments, the clinical intervention is selected from the group consisting of: surgical resection, chemotherapy, radiotherapy, immunotherapy, adjuvant therapy, neoadjuvant therapy, androgen deprivation therapy, and a combination thereof. In some embodiments, the method further comprises administering the clinical intervention to the subject. In some embodiments, the set of biomarkers comprises quantitative measures of a set of cancer-associated genomic loci. In some embodiments, the set of cancer-associated genomic loci comprises one or more members selected from the group consisting of genes listed in Table 1. In some embodiments, the set of cancer-associated genomic loci comprises 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 1. In some embodiments, the set of cancer-associated genomic loci comprises PTEN, TP53 or RB1. In some embodiments, the set of cancer-associated genomic loci comprises PTEN. In some embodiments, the set of cancer-associated genomic loci comprises FGFR3 or ERBB2. In some embodiments, the set of cancer-associated genomic loci comprises one or more members selected from the group consisting of genes listed in Table 2. In some embodiments, the set of cancer-associated genomic loci comprises 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 2. In some embodiments, the set of cancer-associated genomic loci comprises one or more members selected from the group consisting of genes listed in Table 3. In some embodiments, the set of cancer-associated genomic loci comprises 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 3. In some embodiments, the method further comprises using probes configured to selectively enrich the biological sample for nucleic acid molecules corresponding to a set of genomic loci. In some embodiments, the probes comprise nucleic acid primers. In some embodiments, the probes comprise nucleic acid capture probes. In some embodiments, the probes have sequence complementarity with at least a portion of nucleic acid sequences of the set of genomic loci. In some embodiments, the probes have sequence complementarity with at least a portion of nucleic acid sequences of genes selected from genes listed in Table 1, Table 2, or Table 3. In some embodiments, the probes comprise at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 different probes. In some embodiments, the method further comprises determining a likelihood of the determination of the presence or the absence of the cancer in the subject.

In some embodiments, the method further comprises determining a likelihood of the determination of the presence or the absence of the cancer in the subject. In some embodiments, the method further comprises monitoring the presence or the absence of the cancer in the subject, wherein the monitoring comprises assessing the presence or the absence of the cancer in the subject at each of a plurality of time points. In some embodiments, a difference in the assessment of the presence or the absence of the cancer in the subject among the plurality of time points is indicative of one or more clinical indications selected from the group consisting of: (i) a diagnosis of the cancer, (ii) a prognosis of the cancer, and (iii) an efficacy or non-efficacy of a course of treatment for treating the cancer of the subject.

In some embodiments, the prognosis comprises an expected progression-free survival (PFS) or overall survival (OS). In some embodiments, the set of biomarkers from the cfDNA molecules comprise tumor-associated alterations selected from the group consisting of: copy number alterations (CNAs), copy number losses (CNLs), single nucleotide variants (SNVs), insertions or deletions (indels), and rearrangements. In some embodiments, the set of biomarkers from the cfDNA molecules comprise copy number variation. In some embodiments, the set of biomarkers from the cfDNA molecules comprise copy number losses. In some embodiments, the set of biomarkers from the cfDNA molecules comprise single nucleotide variants.

In some embodiments, the method further comprises determining, among the set of biomarkers, a mutant allele frequency of a set of somatic mutations. In some embodiments, the method further comprises determining a blood copy number burden based on copy number alterations or copy number losses of the set of biomarkers. In some embodiments, the method further comprises determining a circulating tumor DNA (ctDNA) fraction of the cancer of the subject based at least in part on the set of mutant allele frequencies. In some embodiments, the method further comprises determining a tumor mutational burden (TMB) of the cancer of the subject based at least in part on the set of mutant allele frequencies. In some embodiments, the method further comprises determining a plasma tumor mutational burden (TMB) of the cancer of the subject based at least in part on the set of mutant allele frequencies comprising microsatellites. In some embodiments, the method further comprises determining an abnormality score of the cancer of the subject based at least in part on the set of mutant allele frequencies.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows an example workflow for identification of somatic mutations in urinary cell-free DNA.

FIG. 2 shows an example workflow for the detection of variants.

FIG. 3 shows a chart of the sensitivity and mutation allele frequency for an assay.

FIG. 4 shows the concordance between expected MAF and MAF detected by a PredicineCARE assay.

FIG. 5 shows the concordance tissue and urine of genomic alterations detected by a PredicineCARE assay.

FIG. 6 shows a schematic of an example workflow.

FIG. 7 shows a schematic of an example workflow.

FIG. 8 shows a schematic of an example workflow.

FIG. 9 shows a schematic of an example workflow.

FIG. 10 shows a schematic of an example workflow of a bioinformatic pipeline.

FIG. 11 provides a schematic of an example study design.

FIG. 12 shows a heat map of the matched urine NGS and FFPE tissue RT-PCR

FIGS. 13A-13B show scatter Plots for the variant allele frequency (VAF) between matched FFPE and Urine Variants.

FIGS. 14A, 14B-1, 14B-2, 14C, and 14D show a schematic of an example assay workflow. FIGS. 14B-1 and 14B-2 show an illustration of genome-wide CNV detection and CNB calculation.). FIG. 14C-D shows the LP-WGS CNV profiles.

FIGS. 15A-15D show charts relating to analytical evaluation of PredicineCNB on clinical plasma samples.

FIG. 16A shows LP-WGS CNV profile heatmap of 14 non-muscle invasive bladder cancer patient samples.

FIG. 16B shows LP-WGS CNV profile heatmap of 33 muscle invasive bladder cancer patient samples.

FIG. 17A shows CNB score comparisons of FFPE, plasma, and urine samples between non-muscle invasive and muscle invasive/non-organ confined bladder cancer patients.

FIGS. 17B-1 and 17B-2 show a LP-WGS CNV profile of two non-invasive bladder cancer patients before and after TURBT surgery.

FIG. 17C shows LP-WGS gene copy number of key bladder cancer genes before and after TURBT.

FIG. 18 shows an schematic of an example workflow.

FIG. 19 shows the PredicineEPIC library of 1 ng, 2.5 ng, 10 ng input as compared to a standard 50 ng DNA input that is used for a standard whole-genome bisulfite sequencing library.

FIG. 20 shows a graph showing Uniform Manifold Approximation and Projection (UMAP).

FIG. 21 shows an example heatmap of significance scores (color scale) of abnormally methylated fragments.

FIG. 22 shows charts relating to methylation abnormality score.

FIG. 23 shows mutation profiles of urinary and tissue tumor DNA from MIBC.

FIGS. 24A-24D show charts relating to concordance and number of mutations detected from the tDNA and utDNA in the WES regions and ATLAS regions.

FIG. 25 shows plots of tumor fractions in tissue and urine.

FIG. 26A-26C show study schematics, samples collection, and assay timeline and operations for an example workflow.

FIGS. 27A-27B show data from an example PredicineWES+ assay.

FIG. 28A-28B shows a series of bCNB scores during treatment for multiple patients.

FIG. 29 shows the copy number gain and loss of determined via PredicineCNB.

FIG. 30 shows the mutational landscape determined via PredicineWES+.

FIG. 31 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Provided herein are systems and methods for detection of the presence or absence of cancer in a subject. The systems and methods provided herein comprises assaying polynucleotides to identify biomarkers of cancers in a subject. The biomarkers may be processed in order to identify the presence or absence of cancer. The methods described herein may process analytes to determine a presence or absence of cancer. The analytes may comprise cfDNA or other analytes that can be provided via non-invasive methods. By analyzing analytes obtained by non-invasive methods, the methods may allow for improved or similar detection or determination of a prognosis as compared to methods that use tumor or tissue biopsy.

Human urine can contain fragmented DNA known as urinary cell-free DNA (ucfDNA) that originates from dying cells in the urogenital tract or from circulating DNA passed through the glomerular filtration. Given the direct access of the urinary tract, urine is a viable source for detecting cfDNA biomarkers and ucfDNA may improve current diagnostic sensitivity for liquid biopsy in genitourinary cancers. Many gene variants can be identified in ucfDNA from cancer patients, particularly from bladder cancer patients. Using urine for liquid biopsy provides a completely noninvasive approach for detection of genomic biomarkers to guide cancer treatment.

Next-generation sequencing has revolutionized cancer genomic research in the last 10+ years. NGS technologies commercially available for guiding treatment plans for cancer patients that have been FDA cleared or approved for use in processing DNA from patient tissue or blood samples. A next-generation sequencing (NGS) assay on cfDNA can enable an accurate detection of genomic alterations, including single nucleotide variant (SNV), insertion and deletion (Indel), Copy number variation (CNV), and DNA re-arrangement. Urine cfDNA originates directly from dying cells exfoliated in urine and can be considered more representative of the tumor than a tissue biopsy due to tumor heterogeneity, since tissue biopsy can only account for mutations found in a specific region of the tumor. In addition, urine can contain fewer contaminating proteins than blood, and the cfDNA level in urine may be greater than in the bloodstream. Urinary cfDNA can be subjected to sequencing to allow detection of cancer and genetic alterations associated with cancer from a subject's urine. Methods and assays described in this disclosure represents an application of the NGS technology and provides a non-invasive, cost-effective, and potentially more sensitive sampling method for patients with cancer, such as genitourinary cancers, including bladder cancer.

In addition to staging and grading of a patient's tumor, tissue biopsy often represent the gold standard in guiding treatment for cancer patients, including qualification for clinical trials or for FDA-approved treatment options (e.g., the QIAGEN Therascreen FGFR RGQ RT-PCR Kit). However, depending on the location of the tumor or the condition of the patient, tumor biopsies can be painful, and the patient may incur risk of complication, whereby medical treatment can become costly. In some cases, tissue biopsy may not be feasible. Less invasive sampling methods remain an unmet clinical need for treatment of bladder cancer patients. A urine cfDNA Assay and the option for liquid biopsy (e.g., from urine) may help fill the void. In addition, there is opportunity for patients to be tested multiple times by urine liquid biopsy as opposed to tissue biopsy. Therefore, liquid biopsy from urine represents a non-invasive and cost-effective method for obtaining patient samples to determine molecular eligibility for certain treatment strategies.

Urine liquid biopsy samples can also improve patient care. In instances where tissue biopsy is not feasible for bladder cancer patient NGS testing, bladder cancer patients can be monitored using urine liquid biopsy. The assays can identify FGFR molecular eligibility for certain therapeutic products. Additionally, the assays can detect other gene mutations in urine samples from bladder cancer patients, including but not limited to alterations in CDKN2A, HRAS/KRAS, KDM6A, PIK3CA, TERT, TP53 and TSC1, which if identified may help to inform patient treatment.

Bladder cancer is the tenth most common malignancy worldwide, with an estimated 550,000 new cases and 200,000 deaths reported in 2018. The majority of bladder cancer cases are non-muscle invasive bladder cancer (NMIBC), requiring intensive regimens of frequent monitoring, local resection (transurethral resection of bladder tumor [TURBT]), and intravesical therapies to reduce the risk of both recurrent and progressive disease. Despite these efforts, within 5 years 45% to 60% of patients will experience recurrent disease, and nearly 20% of those with high-risk disease will progress to muscle-invasive tumors requiring radical cystectomy (RC). The natural history of high-risk NMIBC is unpredictable; rates of recurrence vary from 15% to 78% and rates of progression to muscle invasion and metastasis vary from <1% to 45%. Long-term outcomes suggest approximately 20% to 25% of the high-risk NMIBC patients ultimately die from bladder cancer.

Analytes that can be used for tumor diagnosis from urine liquid biopsy include cfDNA, non-coding-RNA, exfoliated tumor cells and proteins. During tumor destruction therapies or during apoptotic and necrotic processes, both healthy and diseased cells may release cfDNA fragments that are typically 100-200 base pairs in length. In patients without disease, the phagocytic cells englobe cellular debris and necrotic cells, and thus there are very low levels of cfDNA. In the case of patients with disease, phagocytosis is compromised, DNA digestion is minimal, and the DNA fragments have a random dimension that could exceed 10,000 base pairs. Therefore, the cfDNA level in a patient with disease is often elevated.

A study of prospectively enrolled blood, urine, and tumor tissue samples from 16 bladder cancer patients presenting with hematuria was conducted to identify a panel of DNA markers using an NGS assay. The study demonstrated that gene mutations in urine supernatant and sediments had better concordance with cancer tissue compared with plasma. A 48-gene panel was employed, and analyses suggested that two combinations of genes for genetic diagnostic modeling of DNA from bladder cancer patients were TERT, FGFR3, TP53, PIK3CA and KRAS for urine supernatant and TERT, FGFR3, TP53, HRAS, PIK3CA, KRAS, and ERBB2. The accuracy of the five-gene panel and the seven-gene panel yielded AUCs of 0.94 (95% CI of 0.91-0.97), and 0.91 (95% CI of 0.86-0.96), respectively. The study concluded that cfDNA from urine has great diagnostic potential for identifying bladder cancer in hematuria patients.

Fibroblast growth factor receptors FGFR1 to FGFR4 are tyrosine kinases that are present in many types of endothelial and tumor cells and have been shown to play an important role in tumor cell growth, survival, and migration as well as in maintaining tumor angiogenesis (Turner 2010). There are varied mechanisms for FGFR-related oncogenesis, including gene amplification, mutations, and fusions. FGFR3 genetic alterations are found in 60% to 70% of early stage NMIBC. The observed frequency of FGFR3 alterations in bladder cancer changes with tumor stage and grade. For example, in a prospective cohort of 772 patients with NMIBC, TaG1 (158/257) and TaG2 (139/239) tumors displayed similar mutation frequencies of 61.5% and 58.1%, respectively. FGFR3 mutation was an independent predictor of recurrence in patients with low grade Ta tumors. The mutation frequency was lower among TaG3 (30/88; 34.1%), T1G2 (7/26; 26.9%), and T1G3 tumors (20/119; 17%).

A more recent study with a pooled dataset of matched clinical and genomic data for 263 patients with stage pT1 demonstrated that FGFR alterations were frequent in high-risk patients (39% mutations, 6% fusions, not mutually exclusive). Additionally, this study, distinct from previous reports, demonstrated that the prognosis for patients with FGFR alterations was not different from those without FGFR alterations (Breyer 2020). The prevalence of FGFR alterations appears to be somewhat lower in MIBC as compared to NMIBC, with FGFR3 mutation rates of about 15% reported across several studies.

Urine cfDNA (ucfDNA) can be extracted from a urine from a subject and ucfDNA can be subjected to various reactions to allow for sequencing of the ucfDNA. Library construction of ucfDNA can comprise amplification, ligation of adapter or additional sequences and/or labeling with barcodes to generate a sequencing library. Additionally, a ucfDNA or ucfDNA library can be subjected to enrichment using capture probes or amplification primers to enrich for specific sequences of interest from the cfDNA. The library can then be subjected to sequencing reactions to generate sequencing data.

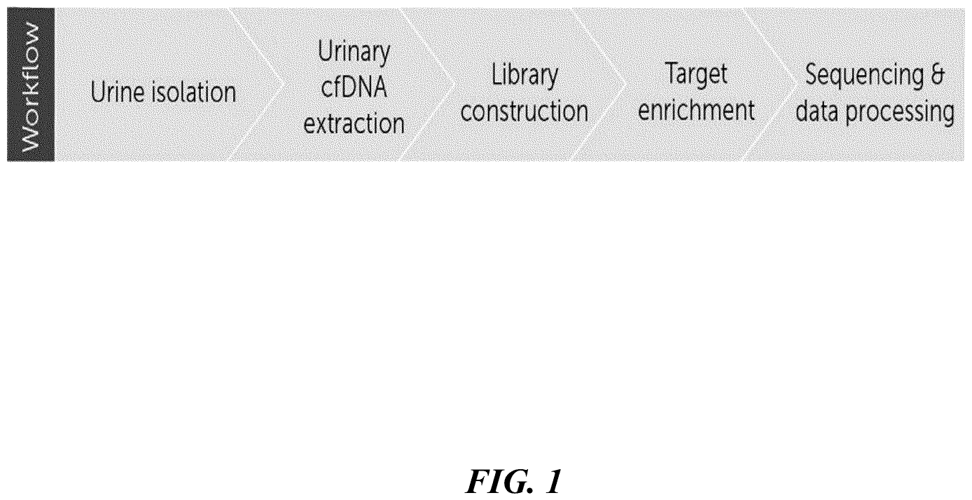

FIG. 1 shows a general workflow of an example assay using urine. A urine sample may be isolated and collected from an individual. Following collection, extraction of urinary cfDNA can performed. Library construction can then be performed on the extracted urinary cfDNA followed by enrichment of specific targets. Once enrichment is performed, the cfDNA can be sequenced and the sequencing data can then be processed.

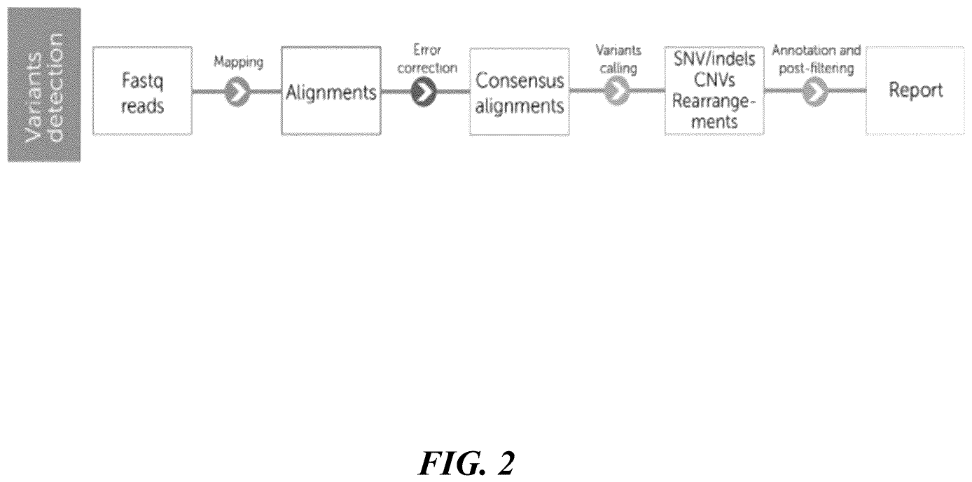

Genetic alterations, such as single nucleotide variations (SNVs), indels, DNA rearrangements, and copy number variations (CNVs) can be identified by bioinformatic analysis of the sequencing data. Generally, a bioinformatic pipeline can utilize the raw sequencing data (e.g., BCL files) and output mutational calls. The pipeline can perform various tasks to analyze the sequencing data such as adapter trimming, barcode checking, or error correction. Cleaned paired files (e.g. FASTQ files) can be aligned to human reference genome using an alignment tool such as BWA alignment tool. Consensus sequences can then be derived by merging paired-end reads that originated from the same molecules as single strand fragments. Single strand fragments from the same double strand DNA molecules can be further merged as double stranded. These processes can allow for sequencing and PCR errors to be corrected. FIG. 2 shows an example bioinformatics analysis workflow.

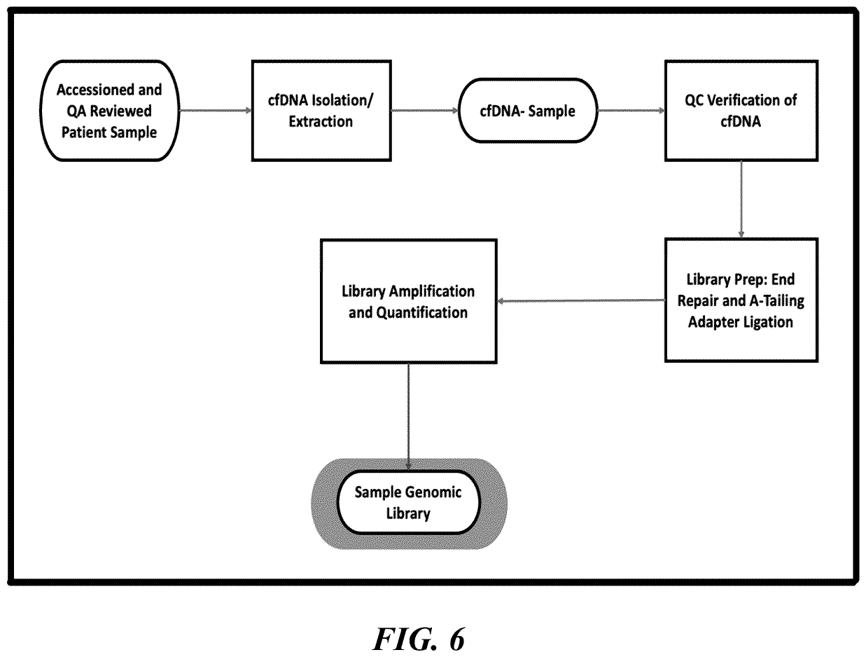

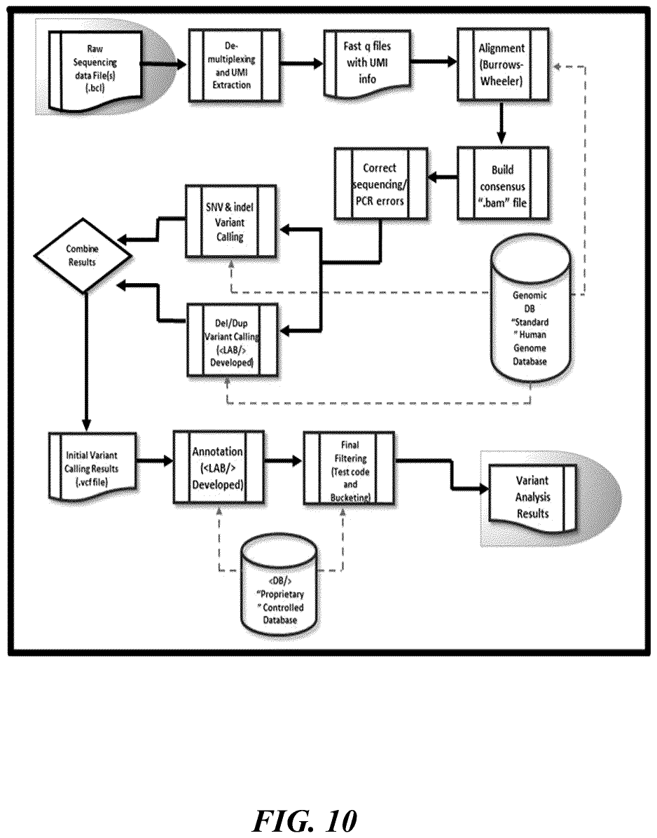

FIG. 6-FIG. 9 illustrate examples of the process workflow of the example urine cfDNA assays (e.g., PredicineCARE). The blue ovals provide connectivity between the diagrams. FIG. 10 illustrates the workflow of bioinformatics pipeline (e.g, DeepSEA). As shown in FIG. 6, a sample may be provided and then the cfDNA can be extracted and isolated from the sample. The cfDNA can then be verified and quality controlled. A sequencing library can be generated from the cfDNA via end repair and A-tailing adapter ligation. The library can then be amplified and quantified.

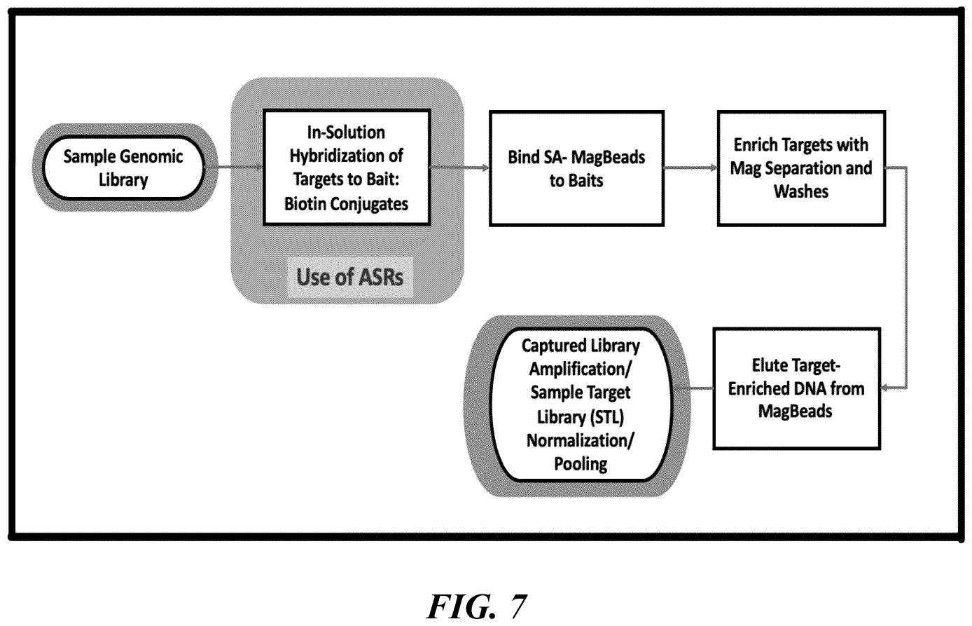

As shown in FIG. 7, once this sample library is generated, the library can be subjected to in solution hybridization with capture probes. The capture probes can be biotinylated. The probes can then be incubated with magnetic beads (e.g., streptavidin magnetic beads) and then subjected to washes to remove any contaminants. The captured DNA can then be eluted from the bead and can be subjected to further amplification, normalization and/or pooling and form a capture library.

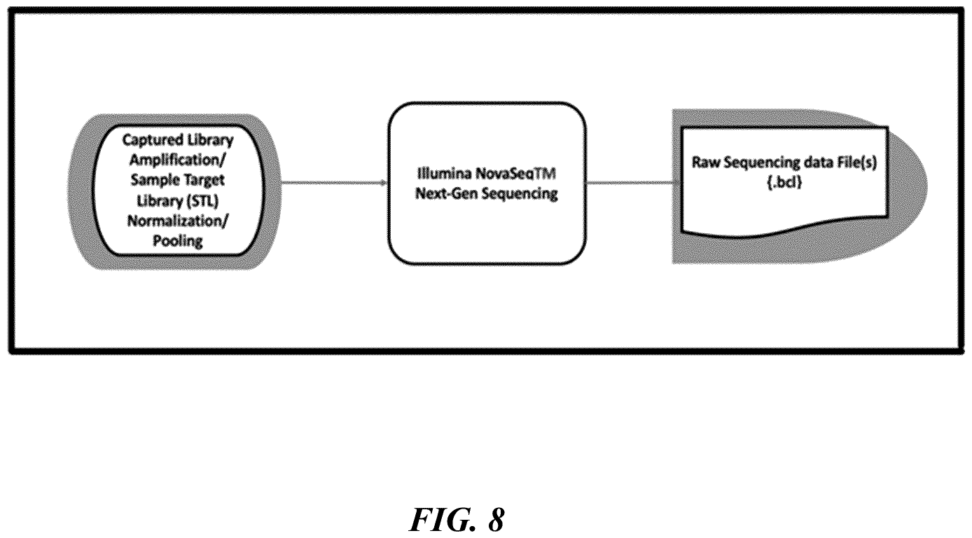

As shown in FIG. 8, the capture library can be sequenced using a next-generation sequencer (e.g., Illumina NovaSeq) and raw sequencing data can be generated.

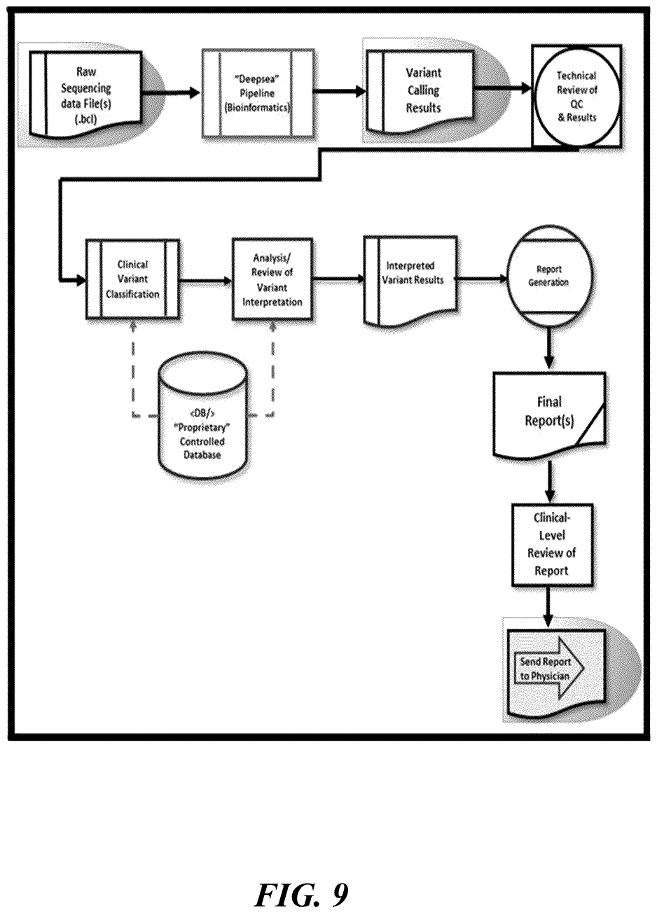

FIG. 9 shows a schematic for raw sequencing processing. The data is fed into a bioinformatics pipeline (e.g., a DeepSea pipeline) to make variant calls. Once the variant calls are made and quality checked, the variants can be classified and analyze to determine the presence of specific variants and can provide information about the presence of a cancer and specific mutations associated with the cancer. This can then be generated into a report and then can be sent to a physician.

FIG. 10 show a schematic of a variant call pipeline (e.g., a DeepSea pipeline). The pipeline can de-multiplex and extract unique molecular identifiers (UMIs) to generate FASTq files with UMI. These files can then be aligned and then a consensus sequence can be generated. Various errors can be corrected based at least in part on analysis of UMIs or the consensus sequence. These error corrected sequencing can then be entered into various variant callers to generate a variant called result. These files can then be annotated and filtered to generate a variant call result.

The subject may be a suspected of a suffering from a cancer. The cancer may be specific or originating from an organ or other area of the subject. For example, the cancer may be breast cancer, lung cancer, prostate cancer, colorectal cancer, melanoma, bladder cancer, non-Hodgkin lymphoma, kidney cancer, endometrial cancer, leukemia, pancreatic cancer, thyroid cancer, and liver cancer, and any combination thereof. The cancer may be a hormone sensitive prostate cancer (HSPC), castrate-resistant prostate cancer (CRPC), metastatic prostate cancer, and a combination thereof. The cancer may comprise biomarkers that are specific to a particular cancer. The specific biomarkers may indicate a presence of a particular cancer. For example, biomarker may indicate that a castrate-resistant prostate cancer is present. The identification of the presence of a type of cancer may allow the determination of a treatment option or recommendation.

In some cases, the subject may be asymptomatic for cancer. For example, the cancer may not exhibit any symptoms and the subject may be unaware of the presence of cancer. The methods described herein may allow a cancer to be identified at an earlier stage than otherwise. The identification of the presence of the cancer at an earlier stage may allow a treatment option or recommendation to be determined at an earlier stage and may allow the subject to have an improved prognosis.

The biological sample may comprise nucleic acids. The biological sample be a cell-free deoxyribonucleic acid (cfDNA) sample or a cell-free ribonucleic acid (cfRNA) sample. The biological sample may comprise genomic DNA or germline DNA (gDNA). The nucleic acid may be a DNA (e.g. double-stranded DNA, single-stranded DNA, single-stranded DNA hairpins, cDNA, genomic DNA, germline DNA, circulating tumor DNA (ctDNA), cell-free DNA (cfDNA)), an RNA (e.g. cfRNA, mRNA, cRNA, miRNA, siRNA, miRNA, snoRNA, piRNA, tiRNA, snRNA), or a DNA/RNA hybrids. The biological sample may be a derived from or contain a biological fluid. For example, the biological sample may be a plasma sample, a serum sample, a buffy coat sample, a peripheral blood mononuclear cell (PBMC) sample, a red blood cell sample, a urine sample, a saliva sample, or other body fluid sample. The biological sample may comprise or be a pleural fluid sample, peritoneal fluid sample, amniotic fluid sample, cerebrospinal fluid sample, lymphatic fluid sample, sweat sample, tear sample, semen sample, or any combination of biological fluid.

The biological sample may be collected, obtained, or derived from the subject using a collection tube. The collection tube may be an ethylenediaminetetraacetic acid (EDTA) collection tube, a cell-free RNA collection tube, or a cell-free deoxyribonucleic acid (DNA) collection tube and CTC collection tubes, or other blood collection tube. The collection tube may comprise additional reagents for stabilizing the nucleic acid molecules or blood cells. The collection tube may allow the nucleic acid or blood cells to be stable such to minimize degradation of the biological sample prior to assaying. The additional reagents may comprise buffer salts or chelators.

The biological sample may be obtained or derived from a subject at various times. The biological sample may be obtained or derived from a subject prior to the subject receiving a therapy for cancer. The biological sample may be obtained or derived from a subject during receiving a therapy for cancer. The biological sample may be obtained or derived from a subject after receiving a therapy for cancer. The biological sample may be collected over 1, 2, 3, 4, 5, 6, 7, 8, 9, 10, 11, 12, 15, 20, 30, 40, 50, 60, 70, 80, 90, 100, 200, 300, 400, 500, 600, 700, 800, 900, 1000 or time points. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more hour period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more day period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more week period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more month period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more year period.

In various aspects as described herein, a clinical intervention or a therapy may be identified at least in part based on the identification of the presences of cancer, or the presence of a parameter of cancer. The clinical intervention may be a plurality of clinical interventions. The clinical intervention may be selected from a plurality of clinical interventions. The clinical intervention may be a surgical resection, chemotherapy, radiotherapy, immunotherapy, adjuvant therapy, neoadjuvant therapy, androgen deprivation therapy, or a combination thereof. In some cases, the clinical interventions may be administered to the subject. After administration of the clinical intervention, a sample may be obtained or derived from the subject such to monitor the cancer or cancer parameters. As such, the methods and systems disclosed herein may be performed iteratively such that monitoring of a cancer can be performed. Additionally, by performing the methods or systems iteratively, therapies or clinical interventions may be updated based on the results of the methods. The monitoring of the cancer may include an assessment as well as a difference in assessment from a previously generated assessment. The difference in an assessment of cancer in the subject among a plurality of time points (or samples) may be indicative of one or more clinical indications such as a diagnosis of the cancer, a prognosis of the cancer, or an efficacy or non-efficacy of a course of treatment for treating the cancer of the subject. The prognosis may comprise expected progression-free survival (PFS), overall survival (OS), or other metrics relating the severity or survivability of a cancer.

The biological samples may be subjected to additional reactions or conditions prior to assaying. For example, the biological sample may be subjected to conditions that are sufficient to isolate, enrich, or extract nucleic acids, such cfDNA molecules.

The methods disclosed herein may comprise conducting one or more enrichment reactions on one or more nucleic acid molecules in a sample. The enrichment reactions may comprise contacting a sample with one or more beads or bead sets. The enrichment reactions may comprise one or more hybridization reactions. For example, the enrichment reactions may comprise contacting a sample with one or more capture probes or bait molecules that hybridize to a nucleic acid molecule of the biological sample. The enrichment reaction may comprise differential amplification of a set of nucleic acid molecules. The enrichment reaction may enrich for a plurality of genetic loci or sequences corresponding to genetic loci. The enrichment reactions may comprise the use of primers or probes that may complementarity to sequences (or sequences upstream or downstream) of a sequence that is to be enriched. For example, a capture probe may comprise sequence complementarity to a set of genomic loci and allow the enrichment of the genomic loci. The enrichments reactions may comprise a plurality of probes or primers. For example, a capture probe may comprise sequence complementarity to a gene selected from Table 1, Table 2, or Table 3. A plurality of probes may comprise 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, or 600 different probes.

The methods disclosed herein may comprise conducting one or more isolation or purification reactions on one or more nucleic acid molecules in a sample. The isolation or purification reactions may comprise contacting a sample with one or more beads or bead sets. The isolation or purification reaction may comprise one or more hybridization reactions, enrichment reactions, amplification reactions, sequencing reactions, or a combination thereof. The isolation or purification reaction may comprise the use of one or more separators. The one or more separators may comprise a magnetic separator. The isolation or purification reaction may comprise separating bead bound nucleic acid molecules from bead free nucleic acid molecules. The isolation or purification reaction may comprise separating capture probe hybridized nucleic acid molecules from capture probe free nucleic acid molecules. The isolation reactions may comprises removing or separating a group of nucleic acid molecules from another group of nucleic acids.

The methods disclosed herein may comprise conduction extraction reactions on one or more nucleic acids in a biological sample. The extraction reactions may lyse cells or disrupt nucleic acid interactions with the cell such that the nucleic acids may be isolated, purified, enriched, or subjected to other reactions.

The methods disclosed herein may comprise amplification or extension reactions. The amplification reactions may comprise polymerase chain reaction. The amplification reaction may comprise PCR-based amplifications, non-PCR based amplifications, or a combination thereof. The one or more PCR-based amplifications may comprise PCR, qPCR, nested PCR, linear amplification, or a combination thereof. The one or more non-PCR based amplifications may comprise multiple displacement amplification (MDA), transcription-mediated amplification (TMA), nucleic acid sequence-based amplification (NASBA), strand displacement amplification (SDA), real-time SDA, rolling circle amplification, circle-to-circle amplification or a combination thereof. The amplification reactions may comprise an isothermal amplification.

The method disclosed herein may comprise a barcoding reaction. A barcoding reaction may comprise the additional of a barcode or tag to the nucleic acid. The barcode may be a molecular barcode or a sample barcode. For example, a barcode nucleic acid may comprise a barcode sequence which may be a degenerate n-mer. The sequence may be randomly generated or generated such to synthesize a specific barcode sequence. The barcode nucleic acid may be added to a sample such to label the nucleic acid molecules in the sample. The barcodes may be specific to a sample. For example, a plurality of barcode nucleic acids may be added to a sample in which the barcode sequence is the same. Upon barcoding of the nucleic acids, those originating from a same sample may have a same barcode sequence, and may allow a nucleic acid to be identified as belonging to a particular or given sample. A molecular barcode may also be used such that each molecule (or a plurality of molecules) in a same volume have a different molecular barcode. This barcode may be subjected to amplification such that all amplicons derived from a molecule have the same barcode. In this way, molecules originating from a same molecule may be identified. The sequences reads may be processed based on the barcode sequences. For example, the processing may reduce errors or allow a molecule to be tracked. Barcode sequences may be appended or otherwise added or incorporated into a sequence by various reactions, for example an amplification, extension, or ligation reaction, and may be performed enzymatically using a nucleic acid polymerase or ligase. The ligation may be an overhang or blunt end ligation and the barcodes may comprise complementarity to nucleic acids to be barcoded. This complementarity may be a sequence derived from the sample from the subject or may be constant sequence generated via a reaction performed on the nucleic acids in the sample.

In some cases, the biological sample may comprise multiple components. For example, the biological sample may be a whole blood sample. The biological sample may be subjected to reactions such to separate or fractionate a biological sample. For example, a whole blood sample may be a fractionated and cell free nucleic acids may be obtained. The whole blood sample may be fractionated using centrifugation such that blood cells may be separated from the plasma (which may contain cell free nucleic acid). A sample may be subjected to multiple rounds of separation or fractionation.

In various aspects described throughout the disclosure, the nucleic acids may be subjected to sequencing reactions. The sequencing the reactions may be used on DNA, RNA or other nucleic acid molecules. Example of a sequencing reaction that may be used include capillary sequencing, next generation sequencing, Sanger sequencing, sequencing by synthesis, single molecule nanopore sequencing, sequencing by ligation, sequencing by hybridization, sequencing by nanopore current restriction, or a combination thereof. Sequencing by synthesis may comprise reversible terminator sequencing, processive single molecule sequencing, sequential nucleotide flow sequencing, or a combination thereof. Sequential nucleotide flow sequencing may comprise pyrosequencing, pH-mediated sequencing, semiconductor sequencing or a combination thereof. The sequencing reactions may comprise whole genome sequencing, whole exome sequencing, low-pass whole genome sequencing, targeted sequencing, methylation-aware sequencing, enzymatic methylation sequencing, bisulfite methylation sequencing. The sequencing reaction may be a transcriptome sequencing, mRNA-seq, totalRNA-seq, smallRNA-seq, exosome sequencing, or combinations thereof. Combinations of sequencing reactions may be used in the methods described elsewhere herein. For example, a sample may be subjected to whole genome sequencing and whole transcriptome sequencing. As the samples may comprise multiple types of nucleic acids (e.g. RNA and DNA), sequencing reactions specific to DNA or RNA may be used such to obtain sequence reads relating to the nucleic acid type.

The sequencing reactions can be performed at various sequencing depths. The sequencing depths of a sequencing reaction may be selected or modulated. The sequencing reactions may comprise sequencing at a region a depth of at least 1×, 2×, 3×, 4×, 5×, 6×, 7×, 8×, 9×, 10×, 11×, 12×, 13×, 14×, 15×, 16×, 17×, 18×, 19×, 20×, 25×, 30×, 35×, 40×, 45×, 50×, 60×, 70×, 80×, 90×, 100×, 200×, 300×, 400×, 500×, 600×, 700×, 800×, 900×, 1000×, 2000×, 3000×, 4000×, 5000×, 6000×, 7000×, 8000×, 9000×, 10,000×, 20,000×, 30,000×, 40,000×, 50,000×, 60,000×, 70,000×, 80,000×, 90,000, 100,000×, or more. The sequencing reactions may comprise sequencing a region at a depth of no more than 1×, 2×, 3×, 4×, 5×, 6×, 7×, 8×, 9×, 10×, 11×, 12×, 13×, 14×, 15×, 16×, 17×, 18×, 19×, 20×, 25×, 30×, 35×, 40×, 45×, 50×, 60×, 70×, 80×, 90×, 100×, 200×, 300×, 400×, 500×, 600×, 700×, 800×, 900×, 1000×, 2000×, 3000×, 4000×, 5000×, 6000×, 7000×, 8000×, 9000×, 10,000×, 20,000×, 30,000×, 40,000×, 50,000×, 60,000×, 70,000×, 80,000×, 90,000, 100,000×, or less.

In various embodiments, a low pass whole genome sequencing is used to sequence nucleic acids. The low pass whole genome sequence may be performed at an average sequencing depth of at least 1×, 2×, 3×, 4×, 5×, 6×, 7×, 8×, 9×, 10×, or more. The low pass whole genome sequence may be performed at an average sequencing depth of no more than 1×, 2×, 3×, 4×, 5×, 6×, 7×, 8×, 9×, 10×, or less. The low pass whole genome sequencing may be performed at an average depth of between 1× and 2×.

In various embodiments, a sequencing reaction may be performed using a set of personalized or customized probes. The sequencing reaction using a set of personalized or customized probes may be a deep sequencing reaction or ultra-deep sequencing reaction. For example, the sequencing reaction using a set of personalized or customized probes may be performed at an sequencing depth of 50×, 60×, 70×, 80×, 90×, 100×, 200×, 300×, 400×, 500×, 600×, 700×, 800×, 900×, 1000×, 2000×, 3000×, 4000×, 5000×, 6000×, 7000×, 8000×, 9000×, 10,000×, 20,000×, 30,000×, 40,000×, 50,000×, 60,000×, 70,000×, 80,000×, 90,000, 100,000×, or more.

In various embodiments, a whole exome sequencing is used to sequence nucleic acids of a subject. The whole exome sequencing may be performed at a non-uniform depth. For example, certain areas of the exome may be boosted or otherwise sequenced at a greater depth than other regions, or at a greater depth than the average depth of the whole exome sequencing. By sequencing certain regions at a higher depth, genes or regions that are of more interest may be analyzed with higher sensitivity, accuracy, and/or precision. Genes or regions associated with or related to cancer can be sequenced at a greater depth. For example, at least 100, 200, 300, 400, 500, 600, 700, 800, 900, or more genes can be sequenced at a higher depth than the rest of the exome (e.g. average depth of the whole exome sequencing).

The sequencing of nucleic acids may generate sequencing read data. The sequencing reads may be processed such to generate data of improved quality. The sequencing reads may be generated with a quality score. The quality score may indicate an accuracy of a sequence read or a level or signal above a nose threshold for a given base call. The quality scores may be used for filtering sequencing reads. For example, sequencing reads may be removed that do not meet a particular quality score threshold. The sequencing reads may be processed such to generate a consensus sequence or consensus base call. A given nucleic acid (or nucleic acid fragment) may be sequenced and errors in the sequence may be generated due to reactions prior or during sequencing. For example, amplification or PCR may generate error in amplicons such that the sequences are not identical to a parent sequence. Using sample barcodes or molecular barcodes, error correction may be performed. Error correction may include identifying sequence reads that do not corroborate with other sequences from a same sample or same original parent molecules. The use of barcodes may allow the identification or a same parent or sample. Additionally, the sequence reads may be processed by performing single strand consensus calling or double stranded consensus call, thereby reducing or suppressing error.

The methods as disclosed herein may comprise determining allele frequency or other cancer related metric. The methods may comprise a mutant allele frequency of a set of somatic mutation among a set of biomarkers. The mutant allele frequency may be used to determine a circulating tumor DNA (ctDNA) fraction of a cancer of a subject. A plasma tumor mutational burden (pTMB) of a cancer of the subject may be determined based at least in part on the set of mutant allele frequencies. Detection of microsatellite instability may also be used to determine the presence or absence of a cancer or cancer metric. Methylation states may be determined using methods described herein and may be used to identify a presence of a cancer or cancer parameter.

In various aspects, sets of biomarkers are processed and data corresponding to the biomarkers are generated. The sets of biomarkers may comprise quantitative measures from a set of cancer-associated genomic loci. The cancer-associated genomic loci may correspond to a set of genes. The cancer associated genomic loci may comprise one or more genes selected from Table 1. In some case, a set of cancer associated genomic loci comprises 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 1. The cancer associated genomic loci may comprise one or more genes selected from Table 2. In some case, a set of cancer associated genomic loci comprises 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 2.

TABLE 1
List of genes

| | | | | | | | |
|---|---|---|---|---|---|---|---|
| ABRAXAS1 | AKT1 | ALK | APC | AR | ARAF | ARID1A | ATM |
| ATR | BAP1 | BARD1 | BRAF | BRCA1 | BRCA2 | BRIP1 | CCND1 |
| CCNE1 | CD274 | CDH1 | CDK12 | CDK4 | CDK6 | CDKN2A | CHEK1 |
| CHEK2 | CTNNB1 | DDR2 | DNAJB1 | EGFR | EPCAM | ERBB2 | ERBB3 |
| ERCC1 | ERCC2 | ERCC4 | ESR1 | EZH2 | FANCA | FANCC | FANCD2 |
| FANCI | FANCL | FANCM | FBXW7 | FGFR1 | FGFR2 | FGFR3 | GNA11 |
| GNAQ | GNAS | HDAC2 | HRAS | IDH1 | IDH2 | JAK2 | JAK3 |
| KIT | KRAS | MAP2K1 | MAP2K2 | MAPK1 | MDM2 | MET | MLH1 |
| MPL | MRE11 | MSH2 | MSH6 | MTOR | MYC | MYCN | MYD88 |
| NBN | NF1 | NFE2L2 | NPM1 | NRAS | NTRK1 | NTRK3 | PALB2 |
| PDCD1LG2 | PDGFRA | PIK3CA | PMS2 | POLD1 | POLE | PPM1D | PPP2R1A |
| PPP2R2A | PRKACA | PRKD1 | PTEN | PTPN11 | RAD50 | RAD51 | RAD51B |
| RAD51C | RAD51D | RAD54L | RAF1 | RB1 | RECQL | RET | RNF43 |
| ROS1 | RPA1 | SMAD4 | SMO | SPOP | STK11 | TERT | TMPRSS2 |
| TP53 | TP53BP1 | TSC1 | TSC2 | VHL | XRCC2 | XRCC3 | XRCC4 |

TABLE 2
List of genes in PredicineCARE panel
Gene List
PredicinePLUS DNA + RNA Panel
SNV and indel (155 key cancer related genes)

| | | | | | | | | | |
|---|---|---|---|---|---|---|---|---|---|
| ABL1 | BRCA2* | CSF1R | FBXW7 | IDH1 | MET* | NTRK1* | PPP2R1A | RNF43 | TET |
| AKT1 | BTK | CTNNB1 | FGFR1* | DH2 | MLH1* | NTRK2* | PRKACA | ROS1 | TGFBR2 |
| ALK | CALR | CSCR4 | FGFR2* | IKZF1 | MPL | NTRK3* | PRKD1 | RUNX1 | TP53* |
| APC* | CBL | DDR2 | FGFR3* | JAK2 | MSH2* | PALB2* | PTCH1* | SERPINB3* | TSC1 |
| AR* | CCND1* | DICER1 | FGFR4* | JAK3 | MSH6* | PAX5* | PTEN* | SERPINB4* | TSC2 |
| ARAF | CCND2 | EGFR | FLT3 | KCNJ5 | MTOR | PBRM1* | PTPN11* | SF3B1 | U2AF1 |
| ARID1A* | CCND3 | EGFR | FOXL2* | KDM6A* | MUTYH | PDCD1LG2* | RAC1 | SMAD4 | VEGFA* |
| ARID2 | CD274* | EPCAM | GATA2 | KDR | MYC* | PDGFRA | RAD51B* | SMARCA4 | VAGFB |
| ASXL1 | CD79B | ERBB2* | GATA3 | KIT* | MYCN* | PDGFRB | RAD51C | SMARCB1* | VHL |
| ATM* | CDH1* | ERBB3 | GNA11 | KMT2C | MYD88 | PIK3CA | RAD51D | SMO* | WT1 |
| ATR | CDK4* | ERBB4 | GNAQ | KRAS* | NF1* | PIK3CB | RAF1 | SPOP | ZNF148 |
| ATRX | CDK4* | ERRFI1* | GNAS | MAP2K1 | NF2* | PIK3CD | RB1 | SRSF2 | |
| B2M | CDKN2A* | ESR1 | H3F3A | MAP2K2 | NFE2L2 | PIK3R1 | RET | STAG2 | |
| BAP1* | CHEK2* | EZH1 | H3F3B | MAPK1 | NOTCH1 | PMS2 | RHEB | STAT3 | |
| BRAF | CREBBP | EZH2 | HNF1A | MDM2 | NPM1 | POLD1 | RHOA | STK11 | |
| BRCA1* | CRKL* | FANC | HRAS | MED12 | NRAS | POLE | RIT1 | TERT | |

Chemotherapy response related genes (9 genes)

| | | | | | | | | |
|---|---|---|---|---|---|---|---|---|
| CYP2C19 | CYP2D6 | CYP3A4 | DPYD | GSTP1 | MTHFR | UGT1A1 | XPC | XRCC1 |

Copy number alteration detection only (15 genes)

| | | | | | | | | | |
|---|---|---|---|---|---|---|---|---|---|
| AKT2 | AREG | AURKA | BCL2 | CCNE1 | CDKN2B | EREG | FGF19 | FGF3 | FGF4 |
| HGF | IGF1R | IGF2 | TOP2A | PPP2R2A | | | | | |

Detection of Select Rearrangements by Breakpoints: 12 genes (11 genes present above)

| | | | | | | | | | |
|---|---|---|---|---|---|---|---|---|---|
| ALK | FGFR1 | FGFR3 | MET | PDGFRA | RET | CD74 | EGFR | GFR2 | MET |
| NTRK1 | PDGFRB | ROS1 | | | | | | | |

*indicated genes covered by full coding regions

The cancer associated genomic loci may comprise one or more genes selected from Table 3. In some case, a set of cancer associated genomic loci comprises 2-600 genes selected from Table 3. In some case, a set of cancer associated genomic loci comprises 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, or 600 members selected from the group consisting of genes listed in Table 3.

TABLE 3
List of genes for PredicineATLAS

| | | | | | | | |
|---|---|---|---|---|---|---|---|
| ABL1 | ABL2 | ABRAXAS1 | ACVR1 | ACVR1B | ACVR2A | ADARB2 | AKT1 |
| AKT1S1 | AKT2 | AKT3 | ALK | ALOX12B | ALOX5 | AMER1 | ANKRD11 |
| APC | APEX1 | AR | ARAF | AREG | ARFRP1 | ARHGAP35 | ARID1A |
| ARID1B | ARID2 | ARID5B | ASXL1 | ASXL2 | ATM | ATR | ATRX |
| AURKA | AURKB | AXIN1 | AXIN2 | AXL | B2M | BABAM1 | BAP1 |
| BARD1 | BCL10 | BCL2 | BCL2L1 | BCL2L11 | BCL2L2 | BCL6 | BCOR |
| BCORL1 | BCR | BIRC3 | BIRC5 | BLM | BMPR1A | BRAF | BRCA1 |
| BRCA2 | BRD2 | BRD3 | BRD4 | BRIP1 | BTG1 | BTG2 | BTK |
| CALR | CARD11 | CARM1 | CASP8 | CBFB | CBL | CCND1 | CCND2 |
| CCND3 | CCNE1 | CCNQ | CD22 | CD274 | CD276 | CD70 | CD74 |
| CD79A | CD79B | CDC42 | CDC7 | CDC73 | CDH1 | CDK12 | CDK4 |
| CDK6 | CDK8 | CDKN1A | CDKN1B | CDKN2A | CDKN2B | CDKN2C | CEBPA |
| CENPA | CHEK1 | CHEK2 | CIC | CNOT3 | CREBBP | CRKL | CRLF2 |
| CSF1R | CSF3R | CTCF | CTLA4 | CTNNA1 | CTNNB1 | CUL3 | CUL4A |
| CUX1 | CXCR4 | CYLD | CYP17A1 | CYP2C19 | CYP2D6 | CYP3A4 | CYSLTR2 |
| DAXX | DDIT3 | DDR1 | DDR2 | DEPDC5 | DEPTOR | DICER1 | DIS3 |
| DLL4 | DNAJB1 | DNMT3A | DNMT3B | DOT1L | DPYD | DUSP4 | DYRK2 |
| E2F3 | EED | EGFR | EIF1AX | EIF4A2 | EIF4E | ELF3 | ELOC |
| EML4 | EMSY | EP300 | EPAS1 | EPCAM | EPHA3 | EPHA5 | EPHA7 |
| EPHB1 | EPHB4 | ERBB2 | ERBB3 | ERBB4 | ERCC1 | ERCC2 | ERCC3 |
| ERCC4 | ERCC5 | ERCC6 | ERCC8 | EREG | ERF | ERG | ERRFI1 |
| ESR1 | ETV1 | ETV4 | ETV5 | ETV6 | EWSR1 | EXO1 | EZH1 |
| EZH2 | EZR | FAAP100 | FAAP20 | FAAP24 | FAM46C | FANCA | FANCB |
| FANCC | FANCD2 | FANCE | FANCF | FANCG | FANCI | FANCL | FANCM |
| FAS | FAT1 | FBXW7 | FEN1 | FGF10 | FGF12 | FGF14 | FGF19 |
| FGF23 | FGF3 | FGF4 | FGF6 | FGFR1 | FGFR2 | FGFR3 | FGFR4 |
| FH | FLCN | FLI1 | FLT1 | FLT3 | FLT4 | FOXA1 | FOXL2 |
| FOXO1 | FOXP1 | FRS2 | FUBP1 | FUS | FZD1 | FZD10 | FZD2 |
| FZD3 | FZD4 | FZD5 | FZD6 | FZD7 | FZD8 | FZD9 | GATA1 |
| GATA2 | GATA3 | GATA6 | GEN1 | GID4 | GLI1 | GNA11 | GNA13 |
| GNAQ | GNAS | GPS2 | GREM1 | GRIN2A | GRM3 | GSK3B | GSTP1 |
| H3F3A | H3F3B | H3F3C | HDAC1 | HDAC2 | HELQ | HES1 | HEY1 |
| HEYL | HGF | HIST3H3 | HLA-A | HLA-B | HNF1A | HOXB13 | HRAS |
| HSD3B1 | HSP90AA1 | ID3 | IDH1 | IDH2 | IDO1 | IFNG | IFNGR1 |
| IFNGR2 | IGF1 | IGF1R | IGF2 | IGF2R | IKBKE | IKZF1 | IL10 |
| IL2RG | IL7R | INHA | INHBA | INPP4B | IRF1 | IRF2 | IRF4 |
| IRS2 | JAK1 | JAK2 | JAK3 | JUN | KAT6A | KCNJ5 | KDM1A |
| KDM5A | KDM5C | KDM6A | KDR | KEAP1 | KIT | KLF4 | KLHL6 |
| KMT2A | KMT2C | KMT2D | KNSTRN | KRAS | LATS1 | LATS2 | LIG1 |
| LIG4 | LMO1 | LYN | MAD2L2 | MALT1 | MAP2K1 | MAP2K2 | MAP2K4 |
| MAP3K1 | MAP3K13 | MAP3K14 | MAP4K3 | MAPK1 | MAPK3 | MAPKAP1 | MAX |
| MCL1 | MDC1 | MDM2 | MDM4 | MED12 | MEF2B | MEN1 | MERTK |
| MET | MITF | MLH1 | MLH3 | MLST8 | MPL | MRAS | MRE11 |
| MSH2 | MSH3 | MSH6 | MST1R | MTHFR | MTOR | MUTYH | MYB |
| MYC | MYCL | MYCN | MYD88 | MYOD1 | NBN | NEGR1 | NF1 |
| NF2 | NFE2L2 | NFKBIA | NHEJ1 | NKX2-1 | NKX3-1 | NOTCH1 | NOTCH2 |
| NOTCH3 | NOTCH4 | NPM1 | NPRL2 | NPRL3 | NRAS | NSD1 | NSD2 |
| NSD3 | NT5C2 | NTHL1 | NTRK1 | NTRK2 | NTRK3 | NUMB | NUP93 |
| NUTM1 | P2RY8 | PAK3 | PALB2 | PARG | PARP1 | PARP2 | PARP3 |
| PAX5 | PBRM1 | PDCD1 | PDCD1LG2 | PDGFRA | PDGFRB | PDK1 | PDPK1 |
| PGR | PHF6 | PHOX2B | PIAS4 | PIK3C2B | PIK3C2G | PIK3C3 | PIK3CA |
| PIK3CB | PIK3CD | PIK3CG | PIK3R1 | PIK3R2 | PIK3R3 | PIM1 | PIN1 |
| PLEKHS1 | PML | PMS1 | PMS2 | POLD1 | POLE | POLH | POLQ |
| POU2F2 | PPARG | PPM1D | PPP2CA | PPP2R1A | PPP2R2A | PPP3CA | PPP4R2 |
| PPP6C | PRDM1 | PRDM14 | PREX2 | PRKACA | PRKAR1A | PRKCI | PRKD1 |
| PRKDC | PRKN | PTCH1 | PTEN | PTPN11 | PTPRD | PTPRT | QKI |
| RAB35 | RAC1 | RAC2 | RAD18 | RAD21 | RAD50 | RAD51 | RAD51B |
| RAD51C | RAD51D | RAD52 | RAD54L | RAF1 | RARA | RASA1 | RB1 |
| RBM10 | RECQL | RECQL4 | REL | RET | REV3L | RGS1 | RHEB |
| RHOA | RHOB | RICTOR | RIT1 | RNF43 | ROBO1 | ROBO2 | ROS1 |
| RPA1 | RPS6KA3 | RPTOR | RRAGC | RRAS | RRAS2 | RSPO1 | RSPO2 |
| RSPO4 | RUNX1 | RXRA | RYBP | SDC4 | SDHA | SDHAF2 | SDHB |
| SDHC | SDHD | SEM1 | SERPINB3 | SERPINB4 | SESN2 | SETD2 | SF3B1 |
| SGK1 | SH2B3 | SH2D1A | SHOC2 | SHQ1 | SLC34A2 | SLFN11 | SLX4 |
| SMAD2 | SMAD3 | SMAD4 | SMARCA2 | SMARCA4 | SMARCB1 | SMARCD1 | SMO |
| SOCS1 | SOCS3 | SOS1 | SOX10 | SOX17 | SOX2 | SOX9 | SPEN |
| SPOP | SRC | SRSF2 | SRY | SS18 | STAG2 | STAT3 | STAT4 |
| STIL | STK11 | STK19 | SUFU | SUZ12 | SYK | TBC1D7 | TBX3 |
| TEK | TERC | TERT | TET1 | TET2 | TGFBR1 | TGFBR2 | TMEM127 |
| TMPRSS2 | TNFAIP3 | TNFRSF14 | TNFRSF1A | TNK2 | TOP1 | TOP2A | TP53 |
| TP53BP1 | TP63 | TP73 | TRAF2 | TRAF3 | TRAF7 | TSC1 | TSC2 |
| TSHR | TSHZ2 | TYRO3 | U2AF1 | UBE2T | UGT1A1 | VEGFA | VEGFB |
| VHL | VTCN1 | WEE1 | WISP3 | WRN | WT1 | XBP1 | XIAP |
| XPA | XPC | XPO1 | XRCC1 | XRCC2 | XRCC3 | XRCC4 | XRCC5 |
| XRCC6 | YAP1 | ZFHX3 | ZNF148 | ZNF217 | ZNF703 | ZNRF3 | ZRSR2 |

The sets of biomarkers may correspond to genetic aberration of a genetic locus. The genetic aberration may a tumor associated alteration. The genetic aberration may comprise copy number alterations (CNAs), copy number losses (CNLs), single nucleotide variants (SNVs), insertions or deletions (indels), and/or rearrangements. The set of biomarkers may be identified in a variety of nucleic acid types. For example, the tumor associated alteration may be identified in cfDNA. The tumor associated alteration may comprise changes in allelic expression, or gene expression. Methods and systems disclosed herein may allow for gene expression profiling and identification of changes to the expression levels of gene.

In various aspects, the methods may comprise identifying the presence of a cancer or a cancer parameter. The methods may comprises determining a probability or a likelihood of the presence of cancer or a cancer parameter. For example, instead of a binary output indicating a presence or absence, an output may be generated that indicates a probability that subject has cancer. This probability may be determined based on algorithms as described elsewhere herein. Similarly, a probability or likely of response to a particular treatment or a probability of relapse may be outputted.

In various aspects, the sets of biomarkers are processed using an algorithm. The algorithm may be a trained algorithm. The trained algorithms may use the sets of biomarkers as an input and generate an output regarding the presence or absence of a cancer. The output may be specific to a type of cancer or subtype of cancer. For example, the output may indicate the presence of bladder cancer.

The trained algorithm may be trained on multiple samples. For example, the trained algorithm may be trained using at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, 9000, 10000, or more independent training samples. The trained algorithm may be trained using no more 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 300, 400, 500, 600,700, 800, 900, 1000, 1100, 1200, 1300, 1400, 1500, 1600, 1700, 1800, 1900, 2000, 3000, 4000, 5000, 6000, 7000, 8000, 9000, 10000, or less, independent training samples. The training samples may be associated with a presence or an absence of the cancer. The training samples may be associated with a relapse of cancer. The training samples may be associated with cancer that is resistant to a particular drug or treatment. An individual training sample may be positive for a particular cancer. An individual training sample may be negative for a particular cancer. By using training samples, the trained algorithm may be able to detect a cancer, determine a probability of recurrence or relapse of a cancer, or determine if a cancer comprises a set of biomarkers may be resistant to a treatment. The training sample may be associated with additional clinical health data of a subject. For example, additional clinical health data may comprise the gender, weight, height, or levels of metabolites or antibodies in a subjects. Additional clinical health data may comprise indication of other diseases, disorders, or diseases conditions.

The trained algorithms may be trained using multiple sets of training samples. The sets may comprise training samples as described elsewhere herein. For example, the training may be performed using a first set of independent training samples associated with a presence of the cancer and a second set of independent training samples associated with an absence of the cancer. Similarly, a first set may be associated with relapse and a second sample may be associated with the absence of relapse.

The trained algorithm may also process additional clinical health data of the subject. For example, additional clinical health data may comprise the gender, weight, height, or levels of metabolites or antibodies in a subject. Additional clinical health data may comprise indication of other diseases, disorders, or diseases conditions that the subject may suffer from. By using the additional clinical health data, in conjunction with the biomarkers, the trained algorithm may output a presence or absences of cancer, probability of relapse, or resistance to drug treatment, that may be different from the output of an algorithm that does not process additional clinical health.

The trained algorithm may be an unsupervised machine learning algorithm. For example, the unsupervised machine learning algorithm may utilize cluster analysis to identify attributes of interest. The trained algorithm may be a supervised machine learning algorithm. For example, the trained algorithm may be trained with training data such to generate an expected or desired output. The supervised learning algorithm may comprise a deep learning algorithm, a support vector machine (SVM), a neural network, or a Random Forest. Via the machine learning algorithm, the trained algorithm may be able to identify relationships of biomarkers to a particular cancer prognosis or diagnosis. Without the trained algorithm, it may otherwise be difficult to identify relationships of the biomarkers to accurately identify the presence of a cancer or other parameters associated with the cancer.

In various aspects, the systems and methods may comprise an accuracy, sensitivity, or specificity of detection of the cancer or a parameter of the cancer. For example, the methods or systems may comprise detecting the presence or the absence of cancer (or the presence of a parameter of the cancer, such as recurrence, relapse, or drug resistance) in the subject at an accuracy of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a sensitivity of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a specificity of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a positive predictive value of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a negative predictive value of at least about 60%, at least about 70%, at least about 75%, at least about 80%, at least about 85%, at least about 90%, at least about 95%, at least about 98%, or at least about 99%.

### Computer Control Systems

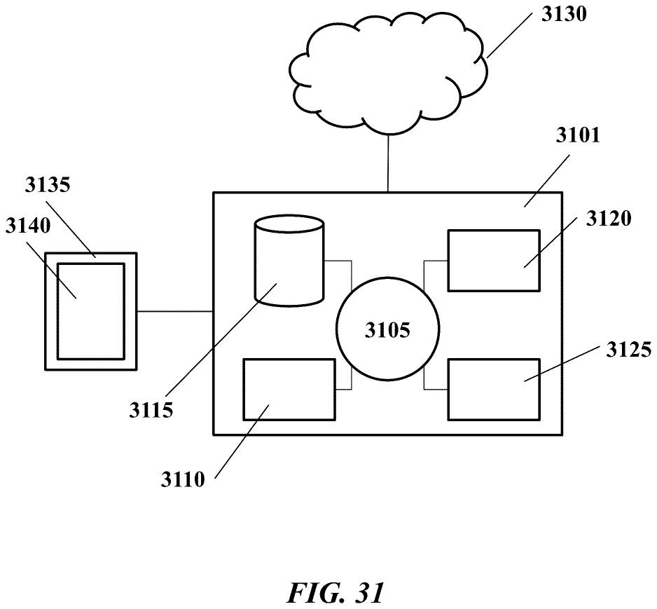

The present disclosure provides computer systems that are programmed to implement methods of the disclosure. FIG. 31 shows a computer system 3101 that is programmed or otherwise configured to perform analysis or operations of the methods, for example determine a likelihood of the presence of a cancer based on a set of biomarkers of an individual or run an algorithm. The computer system 3101 can regulate various aspects of methods and systems of the present disclosure, such as, for example, perform an algorithm, input training data, analyze sets of biomarker, or output a result for the user as to the presence or absence of cancer. The computer system 3101 can be an electronic device of a user or a computer system that is remotely located with respect to the electronic device. The electronic device can be a mobile electronic device.

The computer system 3101 includes a central processing unit (CPU, also “processor” and “computer processor” herein) 3105, which can be a single core or multi core processor, or a plurality of processors for parallel processing. The computer system 3101 also includes memory or memory location 3110 (e.g., random-access memory, read-only memory, flash memory), electronic storage unit 3115 (e.g., hard disk), communication interface 3120 (e.g., network adapter) for communicating with one or more other systems, and peripheral devices 3125, such as cache, other memory, data storage and/or electronic display adapters. The memory 3110, storage unit 3115, interface 3120 and peripheral devices 3125 are in communication with the CPU 3105 through a communication bus (solid lines), such as a motherboard. The storage unit 3115 can be a data storage unit (or data repository) for storing data. The computer system 3101 can be operatively coupled to a computer network (“network”) 3130 with the aid of the communication interface 3120. The network 3130 can be the Internet, an internet and/or extranet, or an intranet and/or extranet that is in communication with the Internet. The network 3130 in some cases is a telecommunication and/or data network. The network 3130 can include one or more computer servers, which can enable distributed computing, such as cloud computing. The network 3130, in some cases with the aid of the computer system 3101, can implement a peer-to-peer network, which may enable devices coupled to the computer system 3101 to behave as a client or a server.

The CPU 3105 can execute a sequence of machine-readable instructions, which can be embodied in a program or software. The instructions may be stored in a memory location, such as the memory 3110. The instructions can be directed to the CPU 3105, which can subsequently program or otherwise configure the CPU 3105 to implement methods of the present disclosure. Examples of operations performed by the CPU 3105 can include fetch, decode, execute, and writeback.

The CPU 3105 can be part of a circuit, such as an integrated circuit. One or more other components of the system 3101 can be included in the circuit. In some cases, the circuit is an application specific integrated circuit (ASIC).

The storage unit 3115 can store files, such as drivers, libraries and saved programs. The storage unit 3115 can store user data, e.g., user preferences and user programs. The computer system 3101 in some cases can include one or more additional data storage units that are external to the computer system 3101, such as located on a remote server that is in communication with the computer system 3101 through an intranet or the Internet.

The computer system 3101 can communicate with one or more remote computer systems through the network 3130. For instance, the computer system 3101 can communicate with a remote computer system of a user (e.g., a medical professional or patient). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 3101 via the network 3130.

Methods as described herein can be implemented by way of machine (e.g., computer processor) executable code stored on an electronic storage location of the computer system 3101, such as, for example, on the memory 3110 or electronic storage unit 3115. The machine executable or machine readable code can be provided in the form of software. During use, the code can be executed by the processor 3105. In some cases, the code can be retrieved from the storage unit 3115 and stored on the memory 3110 for ready access by the processor 3105. In some situations, the electronic storage unit 3115 can be precluded, and machine-executable instructions are stored on memory 3110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 3101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 3101 can include or be in communication with an electronic display 3135 that comprises a user interface (UI) 3140 for providing, for example, an input of biomarkers or sequencing data, or an visual output relating to a detection, diagnosis, or prognosis. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 3105. The algorithm can, for example, determine a presence or absence of a cancer or cancer parameter based on a set of input sequencing data from a sample derived from a subject.

### EXAMPLES

### Example 1: Analysis of Cell Free DNA for Detection of Cancer

Using methods and systems of the present disclosure, circulating tumor DNA in the urine was analyzed to identify genomic alterations for subjects with bladder cancer. Up to 90 mL of urine was collected in tubes containing urine preservative buffer. Upon receipt, samples were accessioned and proceeded immediately into urine supernatant and stored at −80° C. freezer. ucfDNA was extracted and purified by beads then quantified by Qubit, Agilent Bioanalyzer or Fragment Analyzer. Up to 15 ng size-selected ucfDNA can be used for this assay. To construct a library, extracted ucfDNA was labeled with unique molecular barcodes. The ligated sequencing library was PCR amplified with a high-fidelity polymerase and quantified by Bioanalyzer. Target enrichment and hybrid capture was performed. During enrichment, the sequencing library was blocked with adapter specifically blocking oligonucleotides and then hybridized with PredicineCARE panel (e.g., Table 2). Library capture was performed by beads, amplified, and quantified by Bioanalyzer. To perform sequencing, enriched libraries were normalized, pooled, and loaded onto the Illumina platform for 2×150 bp paired-end sequencing. Libraries were sequenced to a median depth of >20,000×.

The PredicineCARE assay identified SNVs, Indels, CNVs, and DNA re-arrangements in one workflow by using a developed bioinformatics pipeline for high accuracy in variant detection. NGS data is analyzed using a DeepSEA NGS analysis pipeline, which starts from the raw sequencing data (BCL files) and outputs the final mutation calls. FIG. 2 shows a schematic of the DeepSEA pipeline. The pipeline performs adapter trimming, barcode checking, and error correction. Cleaned paired FASTQ files were aligned to human reference genome build hg19 using BWA alignment tool. Consensus BAM files were then derived by merging paired-end reads originated from the same molecules as single strand fragments. Single strand fragments from the same double strand DNA molecules were further merged as double stranded. Both sequencing and PCR errors were corrected during this process.

Next a variant filter for SNV and Indel detection was used. Variants were filtered based on variant backgrounds from a pool of normal control samples and other historical samples. Other metrics such as base quality, log odds ratio, and distance to fragment ends were used to remove variants with low confidence. A detected call is a variant with at least 4 unique support fragments of which one should be double-stranded.

Variants were called after filtering low base quality and low mapping score reads. Detected variants were further filtered based on variant background (defined by normal plasma samples and historical data), repeat regions and other quality metrics. Benign and likely benign SNPs were excluded in the variant call list.

A germline variant filter is implemented if a matched normal sample is not available. In real applications, most liquid biopsy assays do not have matched normal. The variant allele frequency was adjusted by copy number changes when it happens at the variant location. Variants annotated in public germline databases, like 1000 genomes, with relative high population allele frequency were also filtered out.

Copy number variation (CNV) was estimated at the gene level. The pipeline calculated the on-target unique fragment coverage based on consensus BAM files then adjusted the GC-bias coverage. The unique fragment coverage profiles from a group of normal samples were used as references to normalize and estimate the z-score of the copy number changes. Re-arrangement was detected by identifying the alignment breaking points based on the BAM files before the consensus operation. Suspicious alignments were filtered based on repeat regions, local entropy calculation and similarity between reference and alternative alignments. Larger or equal to 2 unique alignments were used to report a re-arrangement call.

To obtain a sample, urine was collected using a Urine collection kit. Components of the Urine Collection Kit are listed in Table 4. The kit contains one reagent, which is the Streck® Urine Preserve. Streck Inc. (Omaha, NE, USA) acts as a supplier for the Streck® Urine Preserve reagent in the kit.

TABLE 4
Components of the Urine Sample Collection Kit

| Item | Kit Component |
|---|---|
| 1 | Centrifuge tube, 50 mL |
| 2 | Streck Urine Preserve, 5 mL |
| 3 | Disposal urine cup, 40 mL |
| 4 | Urine collection instruction |
| | pamphlet/instructions for use |
| 5 | Foam lining; generic |
| 6 | Biohazard bag; generic |

To evaluate PredicineCARE panel and assay for somatic alteration detection in ucfDNA, various samples with known mutation alterations including SNV, Indel, DNA re-arrangement, copy number gain, and copy number loss were used for this analytical validation. For this example, the Limit of Detection (LoD) was defined as the lowest mutant allele frequency at which 95% of variants across all replicates for a variant type could be reliably detected by the PredicineCARE assay. Positive percent agreement (PPA) was used to demonstrate assay sensitivity. 15 ng ucfDNA sample input amount was used in the validation study.

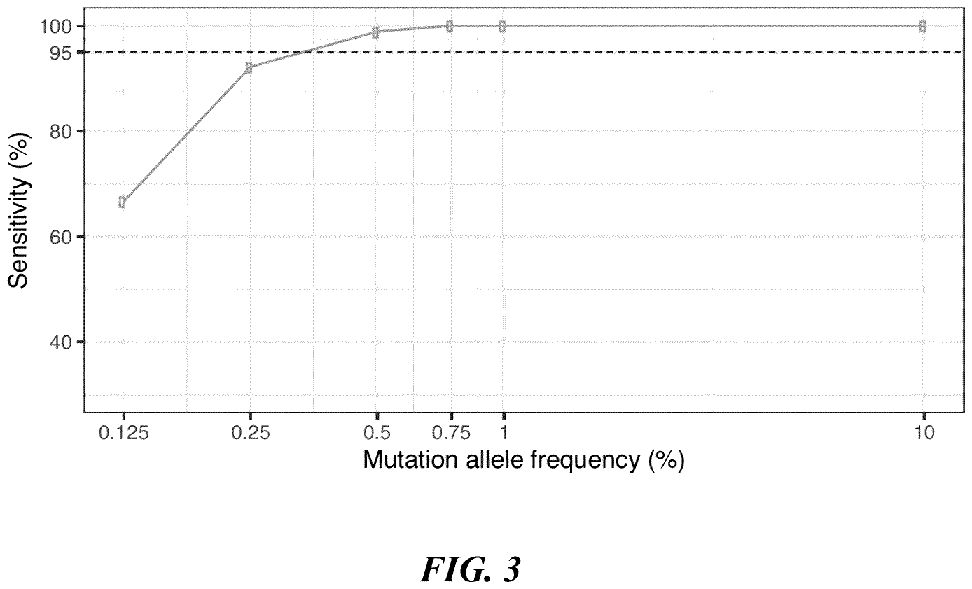

To assess the LoD of single nucleotide variation (SNV) detection in the urine specimen, ucfDNA from a healthy male donor with unique SNPs was spiked into ucfDNA from a healthy female donor, which generated a series of testing materials with variable mutation allele frequencies (MAF) from 0.125 to 10%. 89 mutations were detected at 0.5% MAF out of 90 expected mutations, achieving 98.89% assay sensitivity (95% CI, 94-100%) with 100% PPV (95% CI, 95.9-100%) (Table 5 and FIG. 3).

TABLE 5
Analytical sensitivity of PredicineCARE assay for SNV detection.

| Expected | PPA | PPA 95% | PPV | PPV 95% |
|---|---|---|---|---|
| MAF (%) | (%) | CI (%) | (%) | CI (%) |
| 0.125 | 66.67 | 51-80 | 96.77 | 83.3-99.9 |
| 0.25 | 92.22 | 84.6-96.8 | 94.32 | 87.2-98.1 |
| 0.5 | 98.89 | 94-100 | 100 | 95.9-100 |
| 0.75 | 100 | 96-100 | 100 | 96-100 |
| 1 | 100 | 92.1-100 | 100 | 92.1-100 |
| 10 | 100 | 92.1-100 | 100 | 92.1-100 |

To evaluate the LOD of Indels, CNV, and fusion detection in ucfDNA, we fragmented HD753 gDNA to ucfDNA size and spiked them into ucfDNA from healthy donors, which generated samples with pre-defined allele frequencies (AFs). HD753 reference gDNA from Horizon Discovery (Cambridge, UK) contains known Indels, CNVs, and fusions and was used. For Indels, a total of 35 variants were detected at 0.5 to 1% AF, achieving 97.22% assay sensitivity (95% CI, 85.5-99.9%) and 100% PPV (95% CI, 90-100%) (Table 6).

TABLE 6
Analytical sensitivity of PredicineCARE assay for Indel detection.

| Expected | PPA | PPA 95% | PPV | PPV 95% |
|---|---|---|---|---|
| MAF (%) | (%) | CI (%) | (%) | CI (%) |
| 0-0.5 | 79.41 | 62.1-91.3 | 77.14 | 59.9-89.6 |
| 0.5-1 | 97.22 | 85.5-99.9 | 100 | 90-100 |
| 1-100 | 100 | 47.8-100 | 100 | 47.8-100 |

For copy number variation (CNV) detection, samples with titration levels from 2.375 to 3.125 copies were assessed. All CNV variants were detected at 2.375 copies, achieving 100% assay sensitivity (95% CI, 69.2-100%) and 100% PPV (95% CI, 69.2-100%) (Table 7).

TABLE 7
Analytical sensitivity of PredicineCARE assay for CNV detection.

| Expected copy | PPA | PPA 95% | PPV | PPV 95% |
|---|---|---|---|---|
| number (%) | (%) | CI (%) | (%) | CI (%) |
| 2.375 | 100 | 69.2-100 | 100 | 69.2-100 |
| 2.75 | 100 | 69.2-100 | 100 | 69.2-100 |
| 3.125 | 100 | 63.1-100 | 100 | 63.1-100 |

For DNA re-arrangements, samples with titration levels ranging from 0% AF to 0.825% AF were analyzed. As shown in Table 5, the LoD for DNA re-arrangements detection is 0.25 to 0.55% AF with 100% assay sensitivity (95% CI, 78.2-100%) and 100% PPV (95% CI, 78.2-100%) (Table 8).

TABLE 8
Analytical sensitivity of PredicineCARE assay for fusion detection.

| Expected | PPA | PPA 95% | PPV | PPV 95% |
|---|---|---|---|---|
| MAF (%) | (%) | CI (%) | (%) | CI (%) |
| 0-0.25 | 60 | 14.7-94.7 | 100 | 29.2-100 |
| 0.25-0.55 | 100 | 78.2-100 | 100 | 78.2-100 |
| 0.55-100 | 100 | 69.2-100 | 100 | 69.2-100 |

To evaluate the assay specificity and to ensure that a “blank” sample did not generate analytical signals, 18 ucfDNA samples from healthy donors were tested. Analytic specificity was estimated based on the number of false positive mutations within the targeted panels. Analytic Specificity=100*(1−# of false positives/panel size). Although healthy donors could have low variant frequency pathogenic mutations, like CHIP mutations, we considered all variants passing detection criteria of NGS analysis pipeline as false positives. The analytic specificity is 99.9998%. Precision was measured by the variation of estimated variant frequency between replicates. All samples of the precision assay were assessed from the library construction operation to sequencing analysis.

Repeatability test included intra-run performance (samples processed under the same conditions). Three replicates of each sample were performed at the same condition and results were compared. Reproducibility was assessed based on six samples independently processed under different operating conditions. Concordance of the called variants from replicates is used to assess the precision of intra-assay and inter-assay.

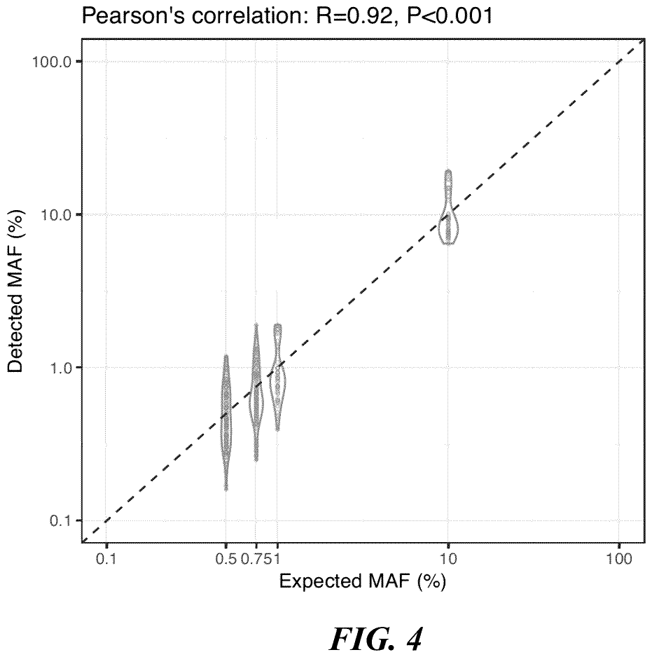

100% concordance between replicates were detected for both intra-run and inter-run precision studies. Furthermore, high concordance was observed between expected MAF and detected MAF in samples of the precision study (Table 9 and FIG. 4).

TABLE 9
100% concordance between replicates were detected
for both intra-run and inter-run precision studies

| | Repeatability | Reproducibility |
|---|---|---|
| Concordant variant number | 90 | 270 |
| Discordant variant number | 0 | 0 |
| Total variant number | 90 | 270 |
| Concordance (%) | 100 | 100 |
| Concordance 95% CI (%) | 96-100 | 98.6-100 |

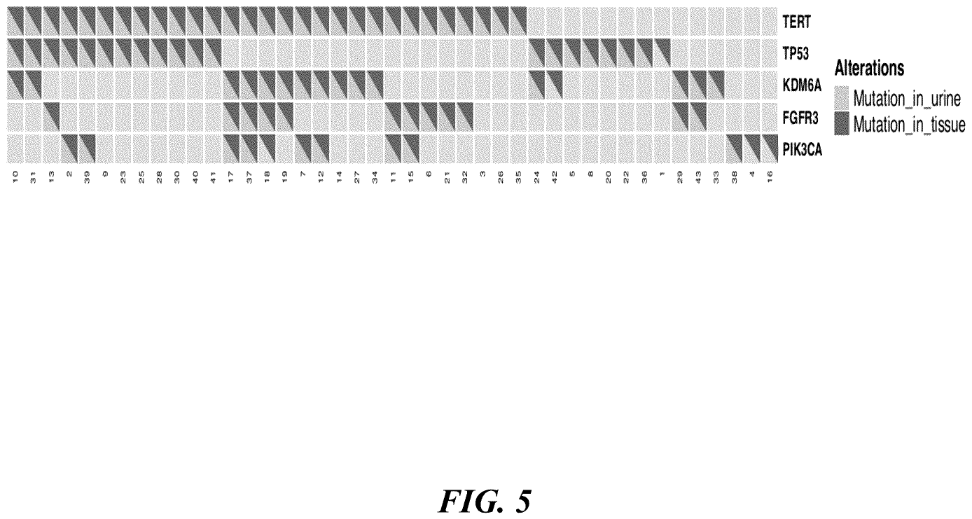

DNA from 43 paired urine and tumor tissue of bladder cancer patients was used to evaluate the performance of urine-based variant detection in clinical samples. The mutations detected in the tissue samples were served as a reference for the mutations detected in ucfDNA. Concordance was determined by comparing the variants detected in urine with those in the paired tissue. The concordance of mutations detected in urine and tissue is 81.0% (95% CI: 77.2-84.4%) (Table 10). Moreover, the PredicineCARE assay achieved 94.6% concordance (95% CI, 87.9-98.2%) for five frequently mutated genes (TERT, TP53, KDM6A, PIK3CA, and FGFR3) in bladder cancer (FIG. 5).

TABLE 10
High concordance of genomic alterations detected
by PredicineCARE assay in paired urine and tissue
samples from bladder cancer patients.

| | Number of | Number of | Number of | | |
|---|---|---|---|---|---|
| | mutations | mutations | concor- | Concor- | Concor- |
| Number of | detected | detected | dant | dance | dance |
| patients | in tissue | in urine | mutations | (%) | 95% CI (%) |
| 43 | 232 | 252 | 196 | 81.0% | 77.2-84.4% |

PredicineCARE assay described in this example analyzed somatic variants in 152 cancer-related genes from liquid biopsy. The assay has undergone rigorous analytical validation testing and demonstrated robust and reproducible results in plasma cfDNA. As demonstrated in this study PredicineCARE assay can detect gene variants in ucfDNA with high sensitivity and specificity (Table 11), which makes the assay an invaluable tool for analyzing somatic variants in various body fluids. Coupled with the completely noninvasive sample collection and the ease of use provided by urine-based liquid biopsy, the PredcineCARE assay offers great potential to enable real-time genomic profiling for cancer detection and to permit more frequent monitoring.

TABLE 11
Summary of PredicineCARE assay performance
for variant detection in urinary cfDNA.

| | Assay | Passing |
|---|---|---|
| Metrics | performance | criteria |
| Precision (intra) | 100% | >95% |
| Precision (Inter) | 100% | >95% |
| LoD | SNV: 0.5% MAF (Sensitivity: 98.89%) | >95% |
| | Indel: 0.5-1% (Sensitivity: 100%) Re- | |
| | arrangement: 0.25-0.55% MAF | |
| | (Sensitivity: 100%) CNV: 2.375 copies | |
| | (Sensitivity: 100%) | |
| Specificity | 99.9998% | >99.9% |

### Example 2: Detection of Bladder Cancer Using Urine

In this example, a study was conducted to evaluate the concordance between tissue tumor DNA profiling and urine cfDNA or circulating tumor DNA (ctDNA) using the PredicineCare Urine cfDNA Assay. The study prospectively enrolled 59 cases of bladder cancer with pathologically confirmed disease and matched tissue/urine paired samples. Baseline peripheral blood mononuclear cell (PBMC) and plasma specimens were collected during visits (Zhang, et al. 2021). The urine, tissue, PBMC, and plasma samples were processed with the PredicineCARE assay and analyzed with the DeepSEA bioinformatics pipeline. Concordance analysis was performed using tissue tumor DNA as reference. Urine cfDNA achieved a specificity of 99.3%, a sensitivity of 86.7% and diagnostic accuracy of 99.1%. FGFR3 alteration and ERBB2 amplification were identified in urine cfDNA. Quantitative metrics including cancer cell fraction, variant allele frequency, and tumor mutation burden were concordant between tumor tissue DNA and urine cfDNA. plasma cfDNA was not highly concordant with tumor tissue DNA due to ctDNA aberrations stemmed from clonal hematopoiesis.

### Example 3: Detection of Bladder Cancer Using a Urine Based NGS Assay

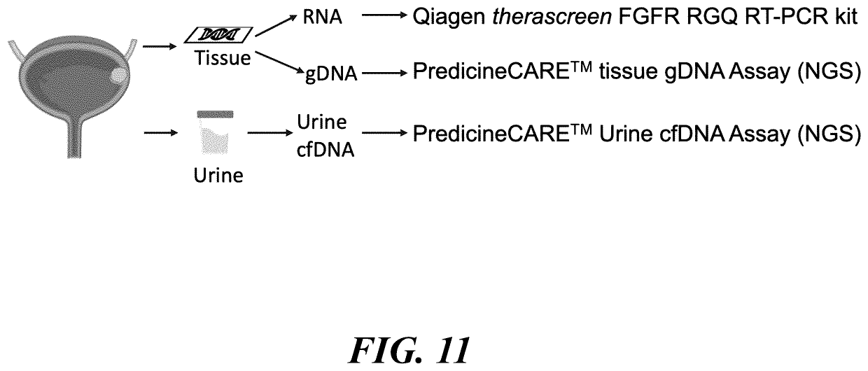

The clinical performance of a urine-based NGS assay was assessed by contrasting the results with those from a FDA-approved tissue-based PCR CDx assay that detects key alterations in FGFR genes. FIG. 11 provides a schematic of the study design. Paired urine and tissue samples were collected from 107 (muscle and non-muscle invasive) bladder cancer patients from the Bladder BRIDGister clinical trial in Germany. Tissue specimens were analyzed using the FDA approved Qiagen therascreen FGFR RGQ RT-PCR kit while matched urine samples were processed using the PredicineCARE™ urine (cell-free DNA) cfDNA NGS assay with a detection sensitivity of 0.3% (0.1% for hotspot mutations). 107 paired bladder cancer urine cfDNA NGS and tissue RT-PCR results were analyzed to determine the concordance (PPA and NPA) between these two assays. A smaller sample set was also used to compare tissue NGS to therascreen RT-PCR and tissue NGS to urine cfDNA NGS. A subset of discordant mutations were additionally validated by ddPCR.

For the concordance analysis between PredicineCARE Urine cfDNA NGS and therascreen FGFR RGQ RT-PCR kit of 107 samples, PPA was 100% (20/20, 95% CI: 83.2-100) and NPA was 94.1% (48/51, 95% CI: 83.8-98.8). PPA and NPA between PredicineCARE Urine cfDNA and Tissue NGS were 100% (19/19, 95% CI: 82.4-100) and 94.2% (49/52, 95% CI: 84.1-98.8) respectively. PPA and NPA between PredicineCARE Tissue NGS and therascreen FGFR RGQ RT-PCR kit were both >95%.

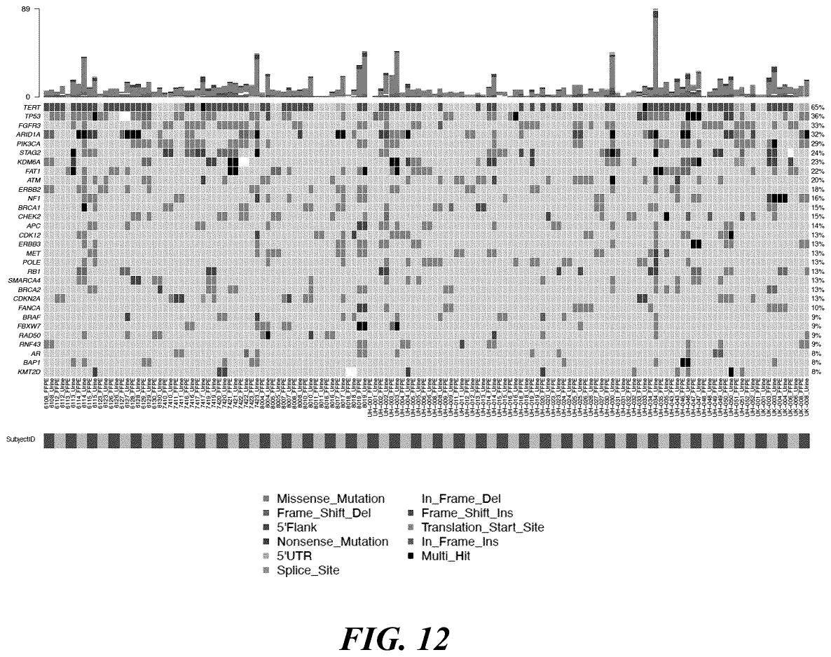

FIG. 12 shows a heat map of the matched urine NGS and FFPE tissue RT-PCR for the identified genetic alterations. Each column represents a sample, and matched FPPE and urine on adjacent columns, with the rows representing a gene. There was a high concordance between Urine NGS and FFPE RT-PCR results. Most samples showed genetic alterations in both Urine NGS and FFPE samples.

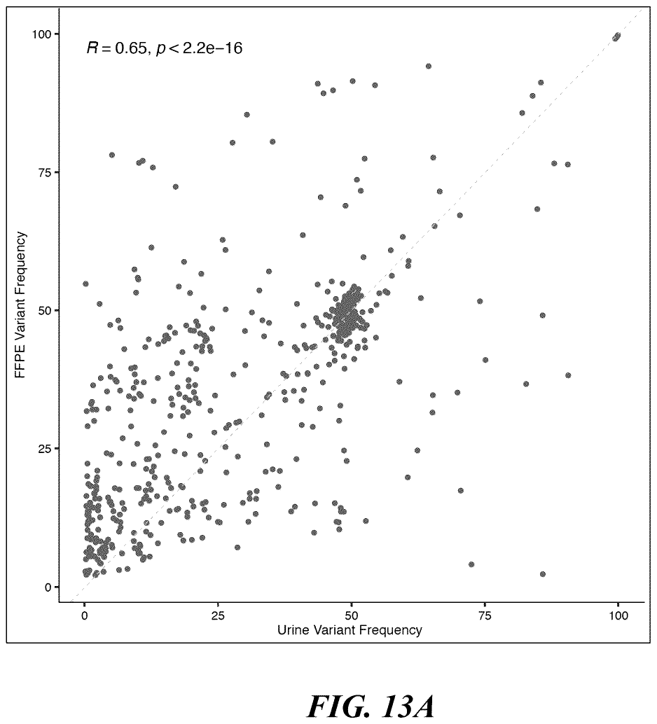

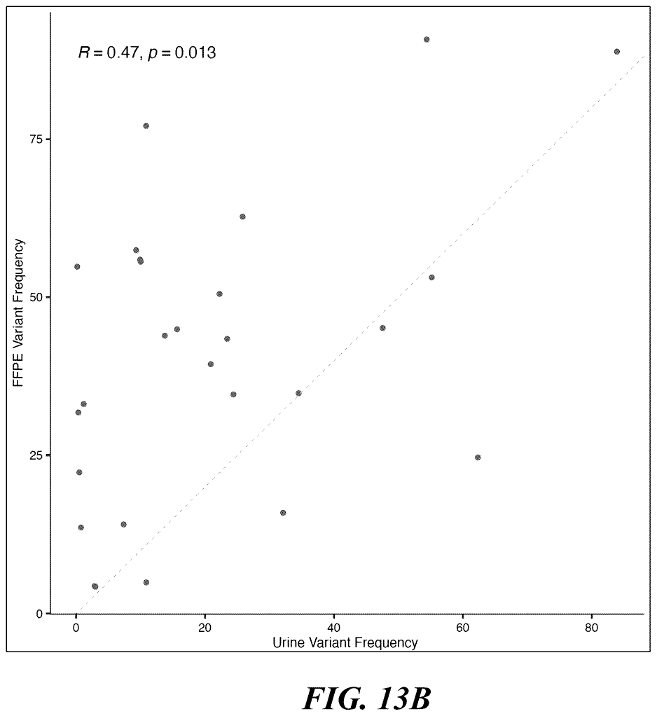

FIGS. 13A-13B show scatter Plots for the variant allele frequency (VAF) between matched FFPE and Urine Variants. FIG. 13A shows a scatter plot for all identified genetic alterations including somatic and germline variants. FIG. 13B shows a scatter plot for somatic FGFR3 alterations. The X axis are VAF of Urine NGS, whereas the Y axis is VAF of FFPE RT-PCR

TABLE 12
Summary of Assay Performance

| | WT* | Mut* | Failed/Invalid | Assay success (%) |
|---|---|---|---|---|
| PredicineCARE | 63 | 27 | 17 | 84.1% (90/107)** |
| Urine cfDNA | | | | |
| Therascreen | 63 | 21 | 23 | 78.5% (84/107)** |
| Tissue RT-PCR | | | | |
| PredicineCARE | 66 | 23 | 6 | 93.6% (89/95)*** |
| Tissue gDNA | | | | |

*WT and Mut: Based on the defined 4 SNVs and 5 Fusions included in the Therascreen RT-PCR assay
**Samples (107) were selected from the matched urine vs tissue sample set for measuring the assay performance
***This is from the comparison between Therascreen RT-PCR vs PredicineCARE Tissue gDNA NGS

Allele frequency (AF) Cut-off of the reportable range for urine NGS (SNV and indels): 0.3% (hot spot: 0.1%)
Allele frequency (AF) Cut-off of the reportable range for FFPE NGS (SNV and indels): 5% (hot spot: 2%)
Allele frequency (AF) Cut-off of the reportable range for fusion: urine (0.1%) and tissue (1%)

TABLE 13
Concordance result between PredicineCARE ™ Urine
NGS and QIAGEN therascreen FGFR RGQ RT-PCR kit

Therascreen Tissue (Reference Method)

| Predicine Urine | FGFR+ | FGFR− | Invalid | Total |
|---|---|---|---|---|
| FGFR+ | 20 | 3 | 4 | 27 |
| FGFR− | 0 | 48 | 15 | 63 |
| Invalid | 1 | 12 | 4 | 17 |
| Total | 21 | 63 | 23 | 107 |

Including invalid urine samples

| | | |
|---|---|---|
| PPA | (20/21) = 95.2% | 95% CI: 76.2-99.9 |
| NPA | (48/63) = 76.2% | 95% CI: 63.8-86.0 |
| OPA | (68/84) = 95.2% | 95% CI: 70.9-88.7 |

Excluding invalid urine samples

| | | |
|---|---|---|
| PPA | (20/20) = 100% | 95% CI: 83.2-100 |
| NPA | (48/51) = 94.1% | 95% CI: 83.8-98.8 |
| OPA | (68/71) = 95.8% | 95% CI: 88.1-99.1 |

Note:
FGFR+ and FGFR− are based on the previously defined 4 SNVs and 5 Fusions included in the Qiagen therascreen FGFR RGQ RT-PCR kit

Three tissue FGFR-negative samples by RT-PCR were FGFR positive by urine NGS testing while 0 urine NGS FGFR negative samples were FGFR positive by tissue RT-PCR (Table 13). Discrepant samples (tissue FGFR WT or invalid but urine positive for FGFR mutations) were further analyzed and confirmed positive by independent orthogonal ddPCR (Bio-Rad ddPCR Mutation Detection Assay) suggesting that discordance between urine and tissue results is often caused by reduced sensitivity of the tissue FGFR RT-PCR test.

High concordance between FGFR alterations detected with an FDA approved tissue companion diagnostic (CDx) assay and urine cfDNA NGS assay demonstrates that the PredicineCARE urine cfDNA NGS assay may represent a novel, accurate, and non-invasive clinical application for molecular diagnostic testing to identify biomarkers in bladder cancer.

### Example 4: Determination of Copy Number Burden Using Low Pass Whole Genome Sequencing

Copy number variation (CNV) is an important characteristic in cancer genome. Blood/Urine-based low-pass whole genome sequencing (LP-WGS) has been increasingly used to identify copy number variations of large genomic ranges in cancer. In this study, we developed PredicineCNB™, a companion LP-WGS assay to robustly estimate genome-wide copy number burden (CNB) from plasma and urine clinical samples, which can provide cancer/normal classification and longitudinal therapy monitoring in a cost-effective way (e.g., using the PredicineSCORE™ liquid biopsy assay (Predicine, Inc., Hayward, CA)).

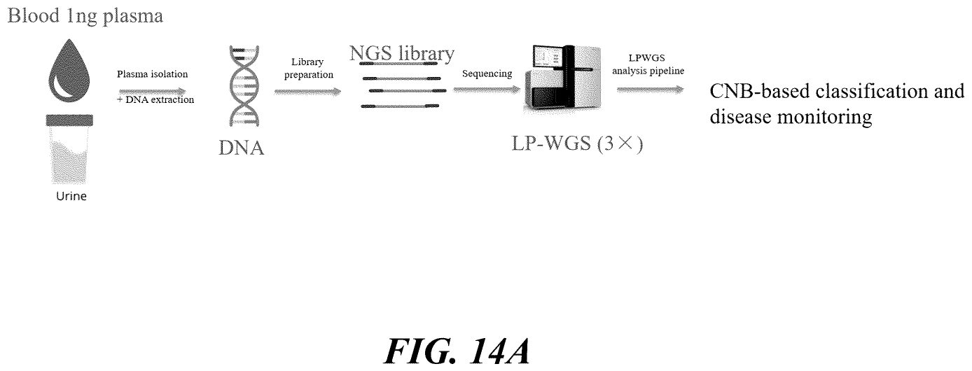

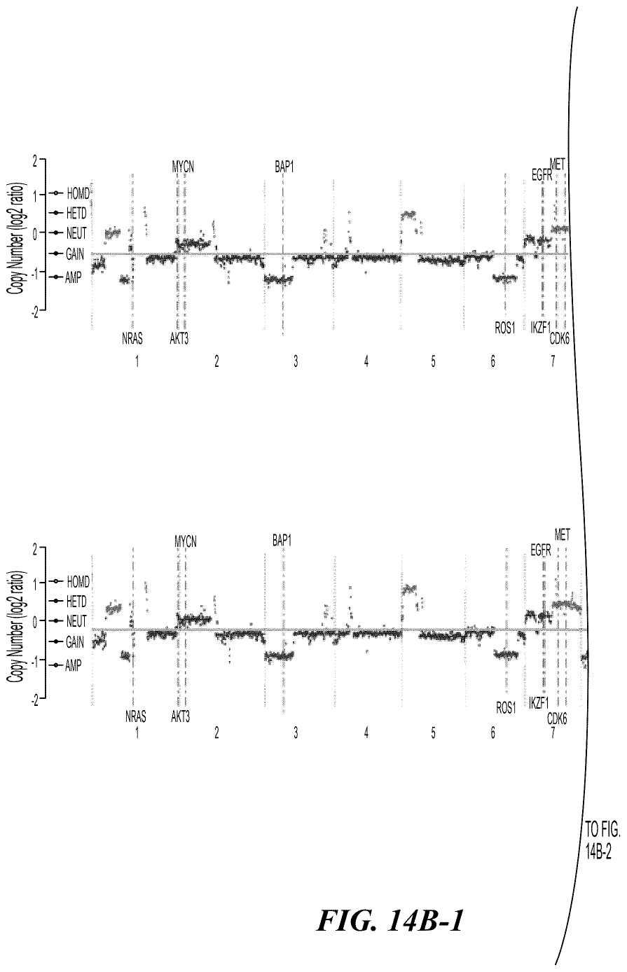

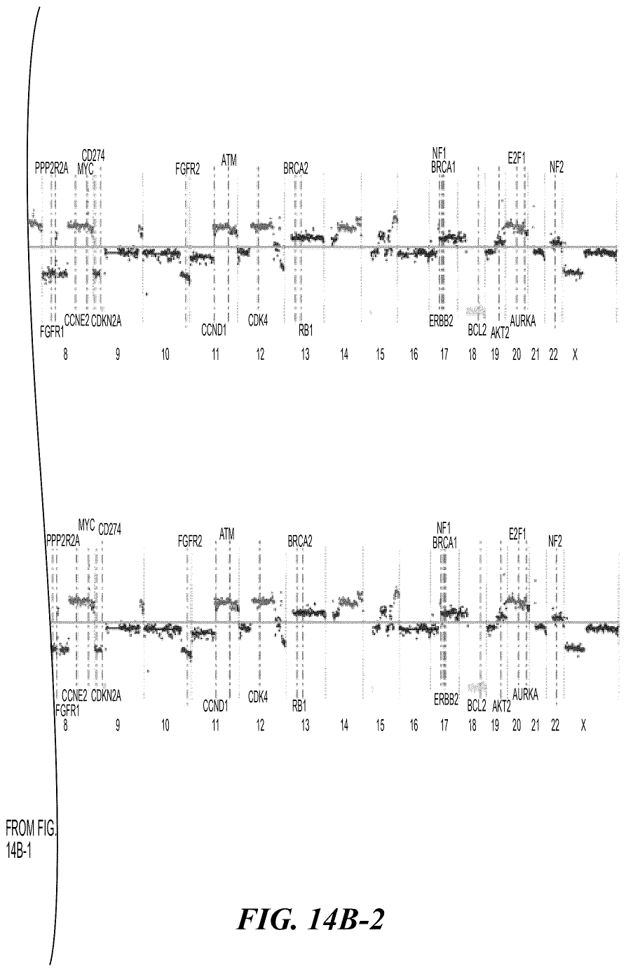

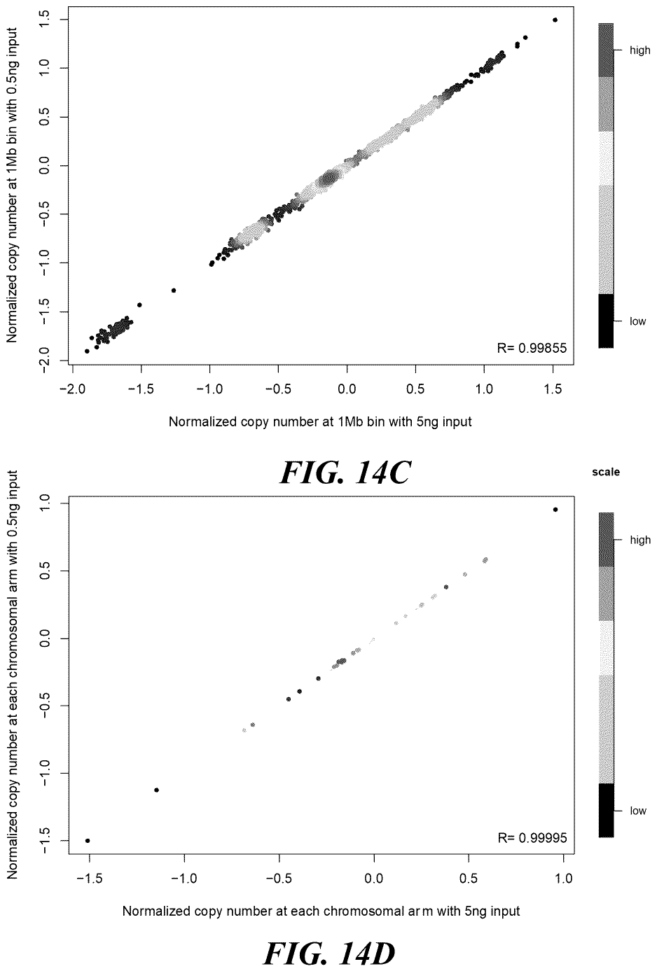

Analytical evaluation was performed based on clinical plasma titration samples, and PredicineCNB™ has demonstrated high cancer detection sensitivity (LOD is 1%) with high specificity, using DNA input amounts as low as 1 ng. FIG. 14A shows a PredicineCNB assay workflow. A blood (or urine sample) is obtained. For a blood sample, the plasma is isolated and the DNA is extracted. The library is then prepared, sequenced using a low pass whole genome sequencing at 3× depth, and then analyzed. FIGS. 14B-1 and 14B-2 show an illustration of genome-wide CNV detection and CNB calculation is robust for small input amount of plasma, as low as 0.5 ng, and shows 5 ng input plasma, CNB score=11.7 (top); 0.5 ng input plasma, CNB score=11.7 (bottom). FIG. 14C shows the LP-WGS CNV profile of 5 ng and 0.5 ng input plasma are highly consistent at 1 Mb region, and FIG. 14D shows LP-WGS copy number between 5 ng and 0.5 ng input plasma are highly consistent at chromosomal arm level, with even higher correlation coefficient than 1 Mb region.

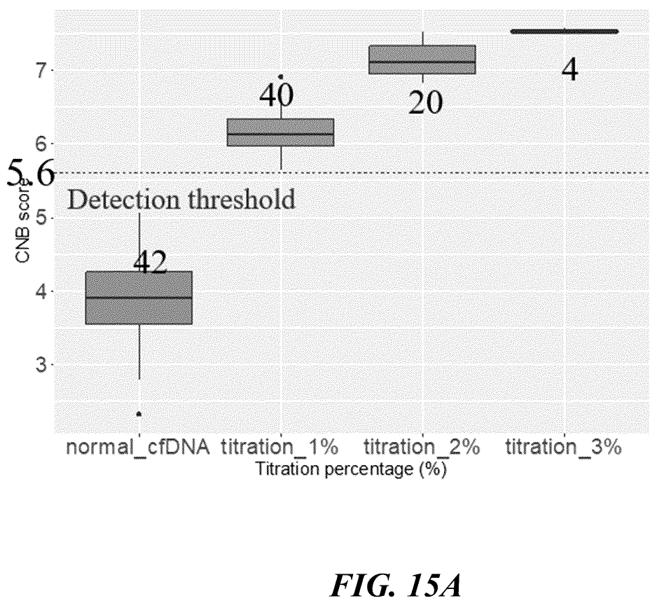

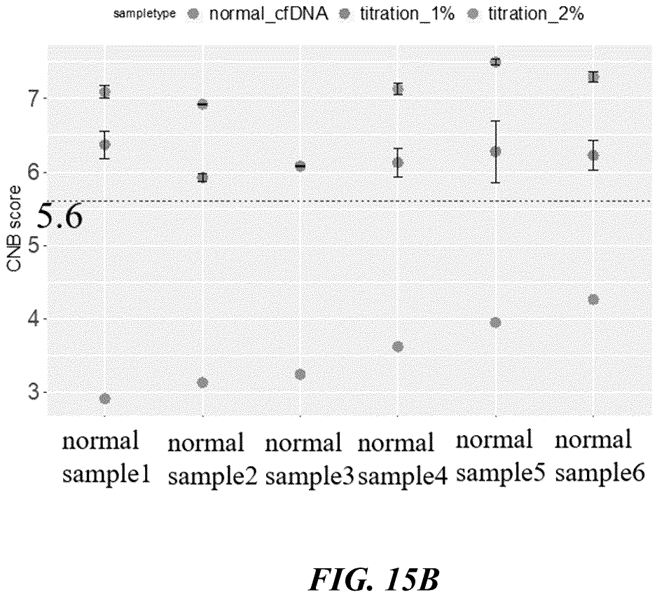

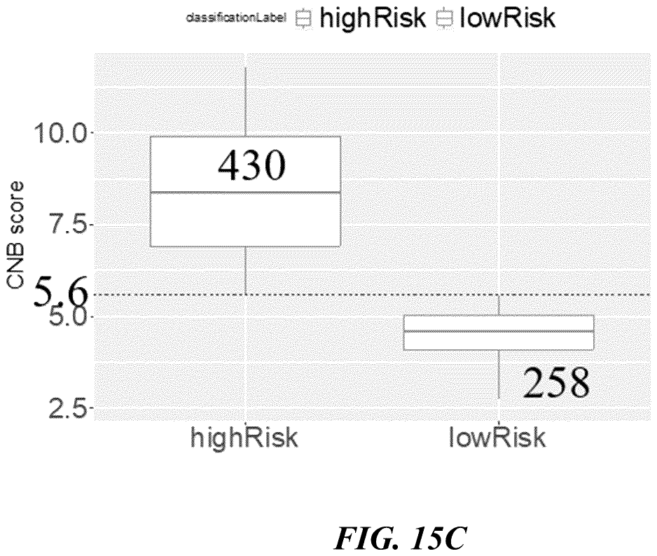

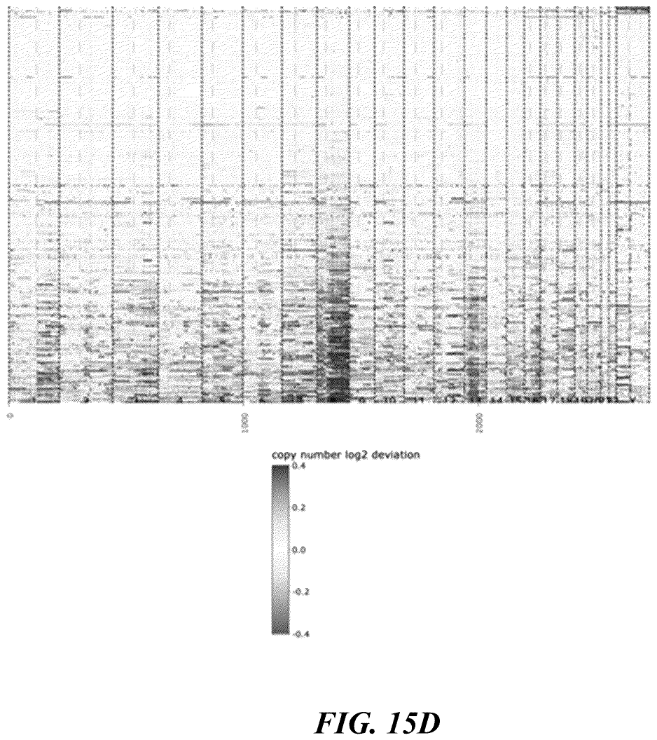

FIGS. 15A-15D show an analytical evaluation of PredicineCNB on clinical plasma samples and application on 688 prostate cancer patient samples. As shown in FIG. 15A, the PredicineCNB LOD is 1% with specificity >97.6% (41/42). The CNB score is generated for cancer plasma titration samples and corresponding normal plasma baseline samples, as shown in FIG. 15B. As shown in FIG. 15C, a CNB score distribution of 688 clinical prostate cancer patients is generated with 430 samples categorized as a high risk and 258 samples categorized as a low risk. FIG. 15D shows the LP-WGS CNV profile heatmap of 430 prostate cancer patient samples classified as high risk, where each row indicates 1 Mb bin copy number deviation across all chromosomes, for each clinical sample.

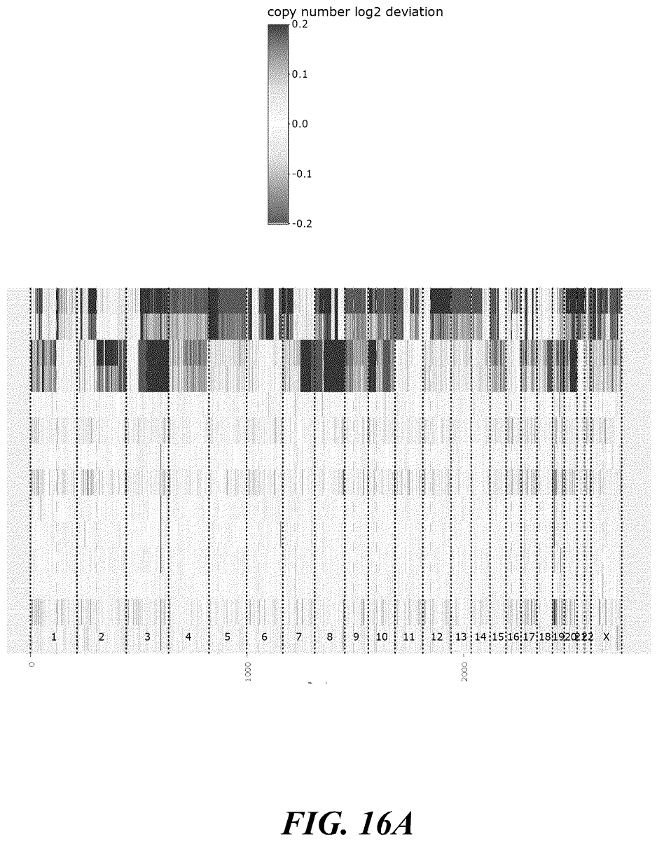

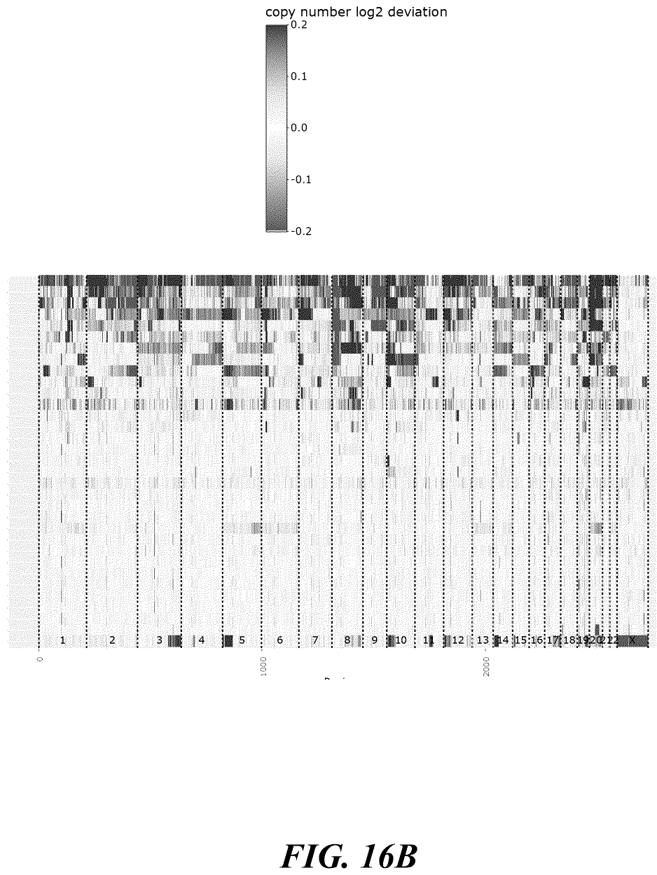

The assay was also performed on non-muscle invasive and muscle invasive bladder cancer patients. FIGS. 16A-16B shows an overview of LP-WGS CNV profile heatmap of non-muscle invasive and muscle invasive bladder cancer patient samples FIG. 16A shows LP-WGS CNV profile heatmap of 14 non-muscle invasive bladder cancer patient samples. FIG. 16B shows LP-WGS CNV profile heatmap of 33 muscle invasive bladder cancer patient samples.

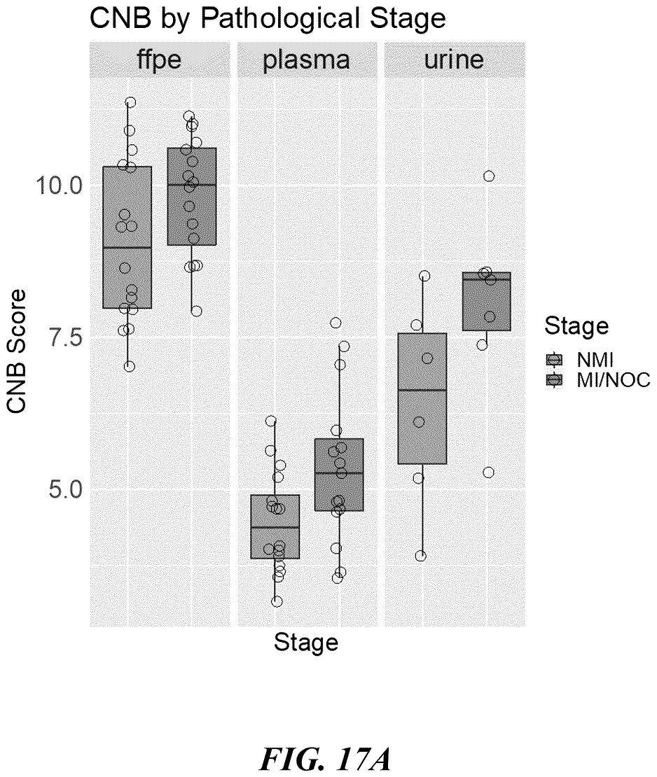

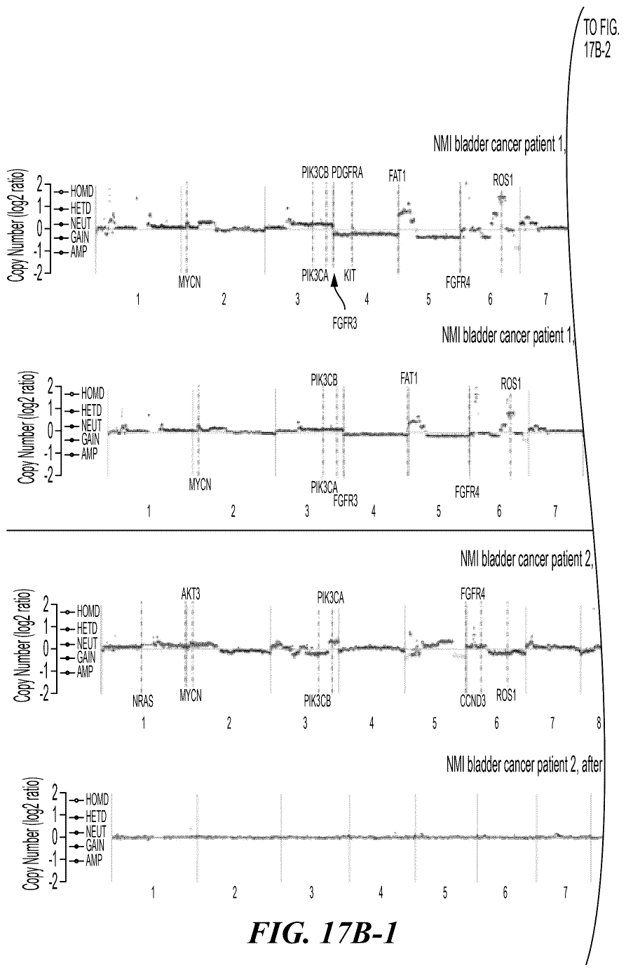

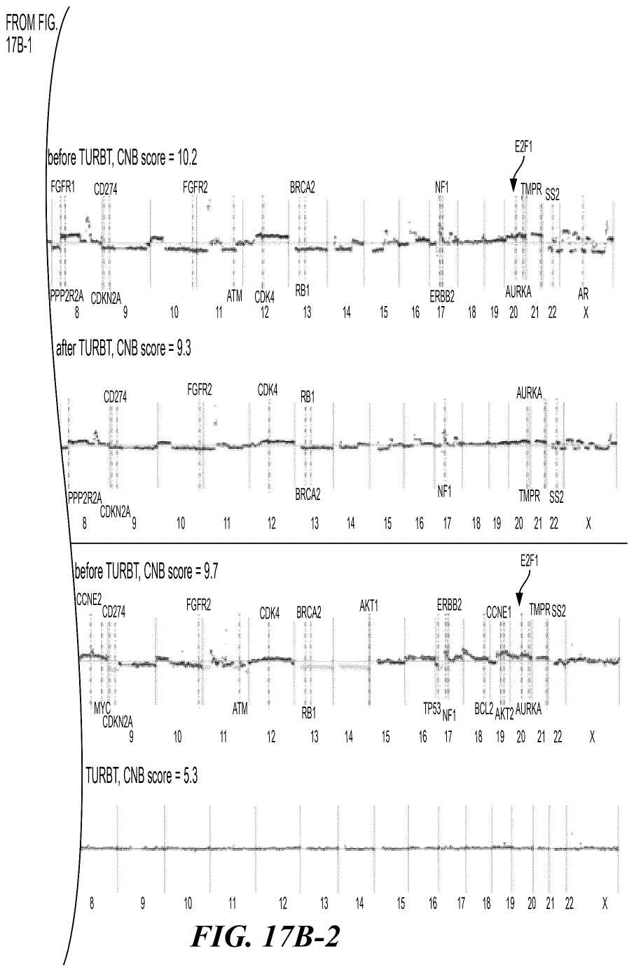

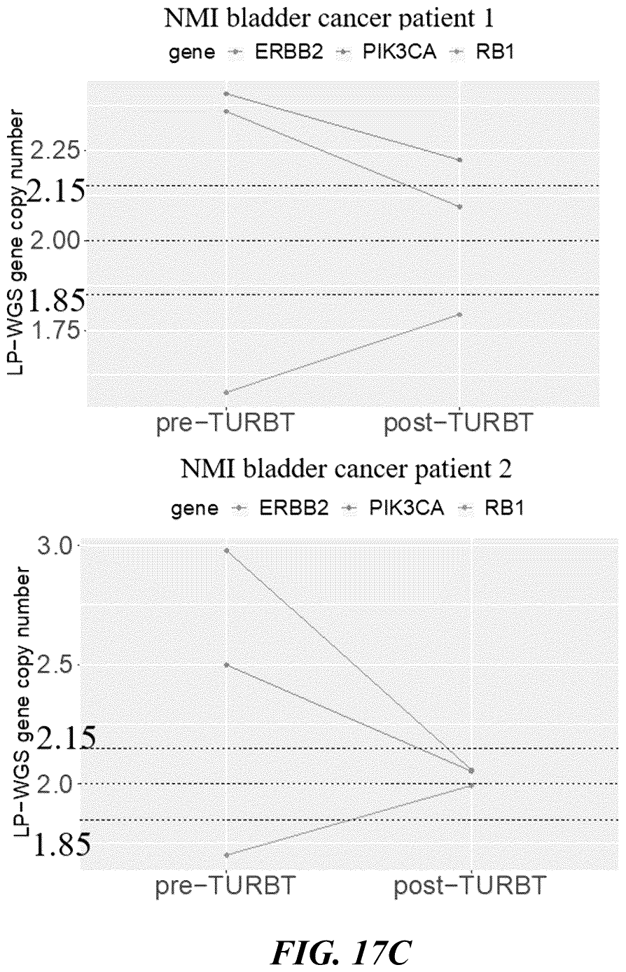

PredicineCNB was tested for usefulness for therapy monitoring of longitudinal bladder cancer patients. FIG. 17A shows CNB score comparisons of FFPE, plasma, and urine samples between non-muscle invasive and muscle invasive/non-organ confined bladder cancer patients. FIGS. 17B-1 and 17B-2 show a LP-WGS CNV profile of two non-invasive bladder cancer patients before and after TURBT surgery, showing a decrease in CNB score for both patients after TURBT surgery. FIG. 17C shows LP-WGS gene copy number of key bladder cancer genes before and after TURBT, demonstrating the monitoring of different cancer genes before and after surgery.

The examples demonstrates an algorithm analysis pipeline for LP-WGS assay. The copy number burden (CNB) LOD is 1% tumor fraction with 1 ng plasma input. PredicineCNB™ demonstrates its high sensitivity of cancer detection, and a promising clinical application of urine and blood-based genome-wide copy number changes in therapy monitoring.

### REFERENCES

[1] Davis A A, Luo J, Zheng T, Dai C, et al. Genomic complexity predicts resistance to endocrine therapy and CDK4/6 inhibition in hormone receptor-positive (HR+)/HER2-negative metastatic breast cancer. Clin Cancer Res. 2023 Jan. 24; CCR-22-2177. doi: 10.1158/1078-0432.CCR-22-2177, which is incorporated by reference herein in its entirety.

### Example 5: Detection and Monitoring Using Methylation

Using methods and systems of the present disclosure, circulating tumor DNA from a biological sample was analyzed. A combined MRD assay was performed using a targeted panel covering hotspot mutations and important genes (Predicine WES+), a companion LP-WGS assay for copy number burden (Predicine CNB), and a whole genome methylation assay (e.g., PredicineEPIC).

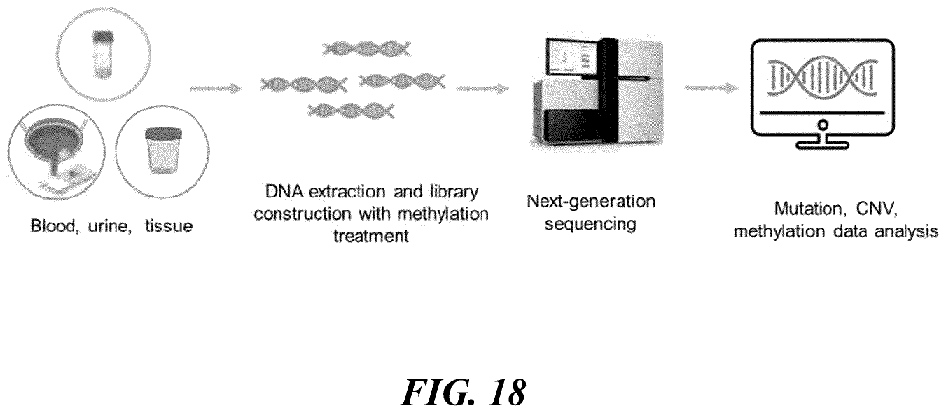

FIG. 18 shows an example schematic of the workflow. A sample (blood, urine or tissue) was obtained from a subject. DNA was extracted and a library was generated. The library was subjected to a methylation treatment. Once the library was constructed, a next generation sequencing was performed, and mutation, CNV, and methylation data were analyzed.

The analytical evaluation was based on titration of real-world clinical patient samples. DNA input amounts ranged from 30 ng down to 1 ng. The clinical evaluation was based on longitudinal samples from patients with different cancer indications, including bladder cancer, mCRPC, CRC, breast cancer and NSCLC.

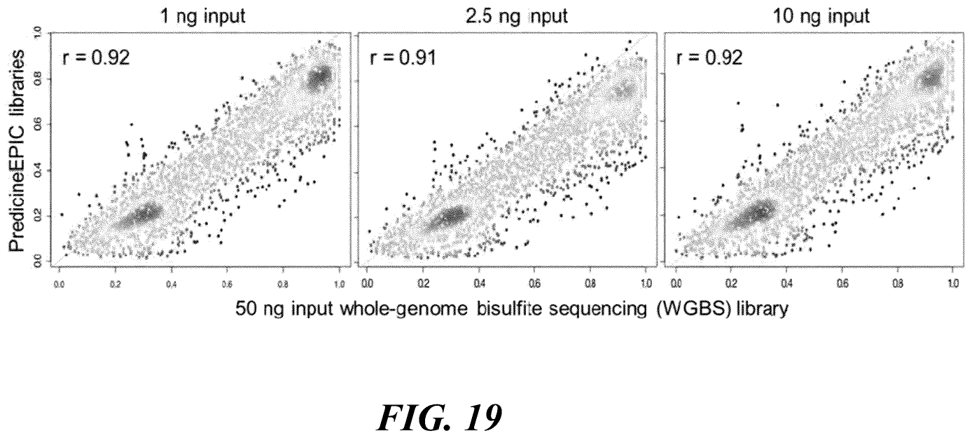

FIG. 19 shows the PredicineEPIC library of 1 ng, 2.5 ng, 10 ng input as compared to a standard 50 ng DNA input that is used for a standard whole-genome bisulfite sequencing library. Specifically, the PredicineEPIC libraries at 1 ng, 2.5 ng and 10 ng provide a similar coverage and data as comparted to a standard 50 ng whole genome bisulfite sequencing library.

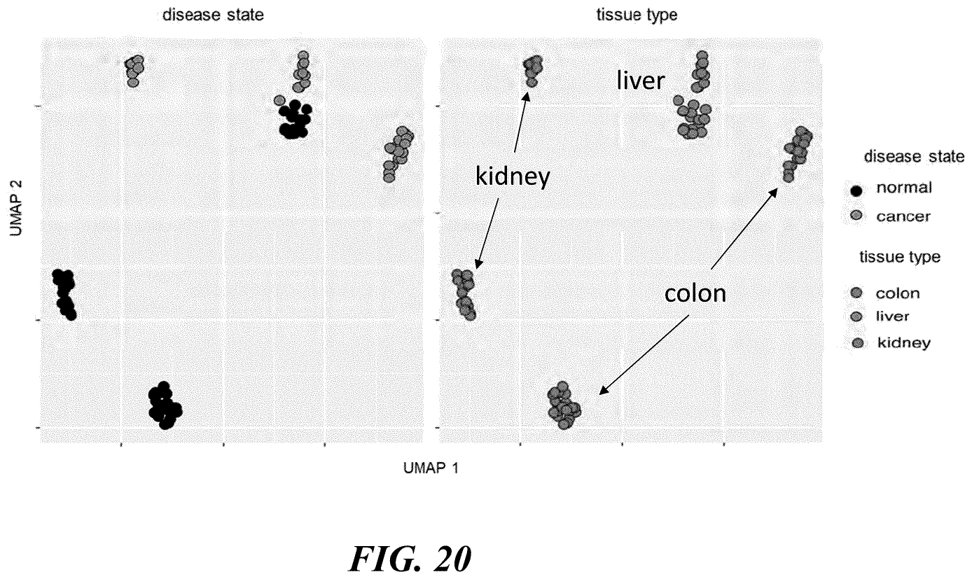

97 cancer and normal tissue types were analyzed using fragment-level DNA methylation analysis. These assays were able to identify fragments of methylation gain or loss as compared to a background model. These assays were able to group different cancers by tissue type and if the sample was cancerous or non-cancerous. FIG. 20 shows a graph showing Uniform Manifold Approximation and Projection (UMAP). Distinct populations are visualized based on differing UMAP scores.

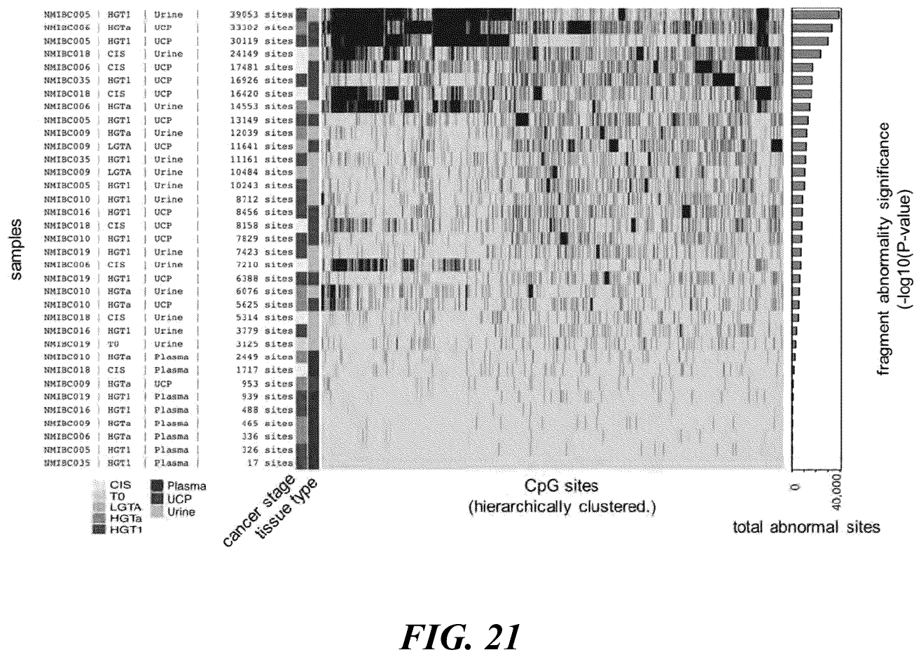

For each sample, a methylation abnormalities score can be calculated based on the methylation data. FIG. 21 shows an example heatmap of significance scores (color scale) of abnormally methylated fragments at 28.4 K of the most variable CpG sites of a total of 142 K covered sites genome-wide (columns) for 35 bladder cancer samples (rows) from patients with different stages and from different sources (row annotations).

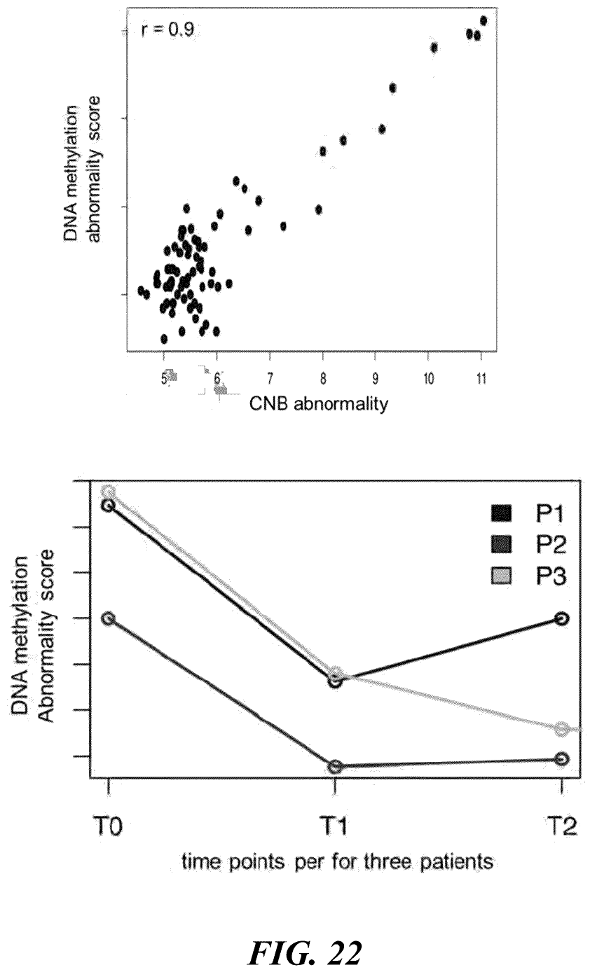

In conjunction with methylation abnormality score, a copy number burden abnormality score can also be calculated on the low pass sequencing data. These two abnormalities scores are shown to be highly correlated as shown in FIG. 22, and can be used in combination to identify a subject as having a cancer. Additionally, a patient's disease progression can be calculated using the DNA methylation score as shown in FIG. 22. Three patients are shows with TO being prior to treatment and T1 and T2 being after treatment with a noticeable decrease in abnormality score subsequent to treatment.

### Example 6: Boosted Whole Exome Sequencing for Deterring of Muscle Invasive Bladder Cancer

Urinary tumor DNA profiling can be used for the diagnosis, monitoring, and treatment stratification of bladder cancer. However, previous studies mainly used the targeted next generation sequencing (NGS) panel approach, which is limited to predefined genes and thus lacks comprehensiveness. Here, this example demonstrates the use of a boosted whole-exome sequencing (WES) to urinary and tissue tumor DNA in muscle-invasive bladder cancer (MIBC) to comprehensively compare the mutation profiles in matched urine and tissue samples.

Matched tumor tissue, urine and peripheral blood mononuclear cells (PBMC) samples were collected from twenty MIBC patients. Nineteen tumor tissue, nineteen urine and twenty PBMC samples passed sample quality control were processed for NGS. PredicineWES+, an NGS assay with whole-exome coverage and boosted coverage in 600 cancer related genes from the PredicineATLAS panel, was applied to matched tumor, urine and PBMC samples for variant profiling. Mutation profiles of tumor tissue and urinary DNA were analyzed and compared.

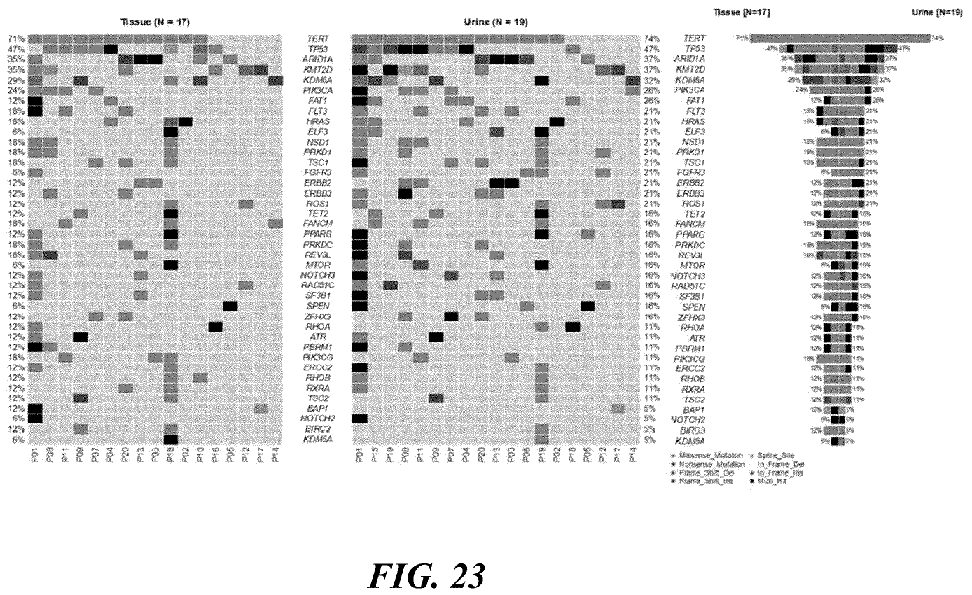

FIG. 23 shows mutation profiles of urinary and tissue tumor DNA from MIBC. Mutation profiles of urinary and tissue tumor DNA were highly concordant across patients, with frequently mutated genes (TERT. TP53, ARID1A, KMT2D, KDMSA, PIK3CA, etc.) displaying comparable prevalence. Two tissue samples with sequencing QC failed were not shown.

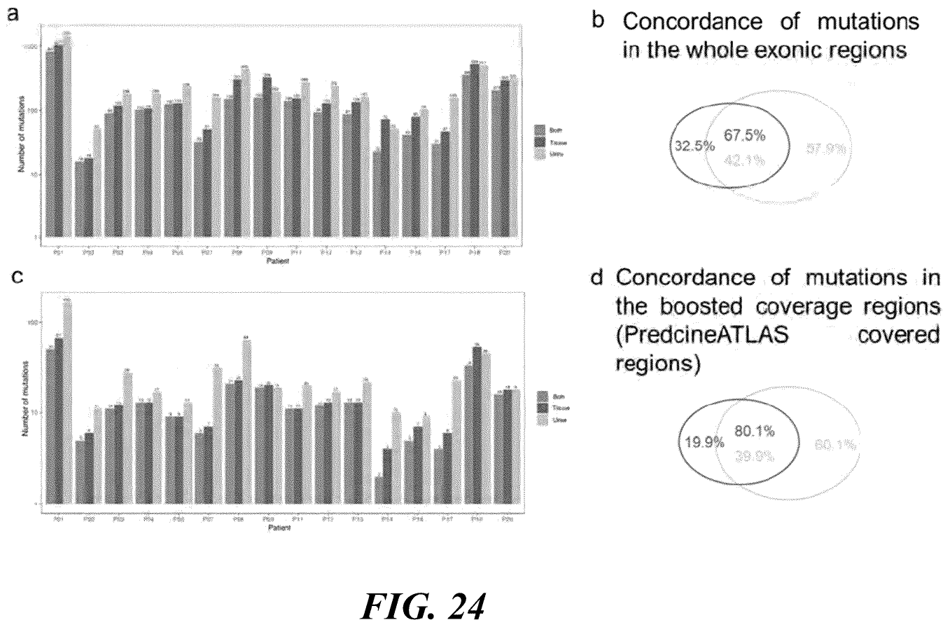

Concordance of the mutations detected from the tissue tumor DNA (tDNA) and urinary tumor DNA (utDNA) by PredicineWES+. Number of mutations detected from the tDNA and utDNA in the WES regions (FIGS. 24A-B) and ATLAS regions (FIG. 24C-D) were compared. Majority of the tDNA mutations (67.5% in WES regions and 80.1° A) in ATLAS regions) were also detected in utDNA. However, less than half of the utDNA mutations (42.1% in WES regions and 39.9% in ATALS regions) were detected in tDNA.

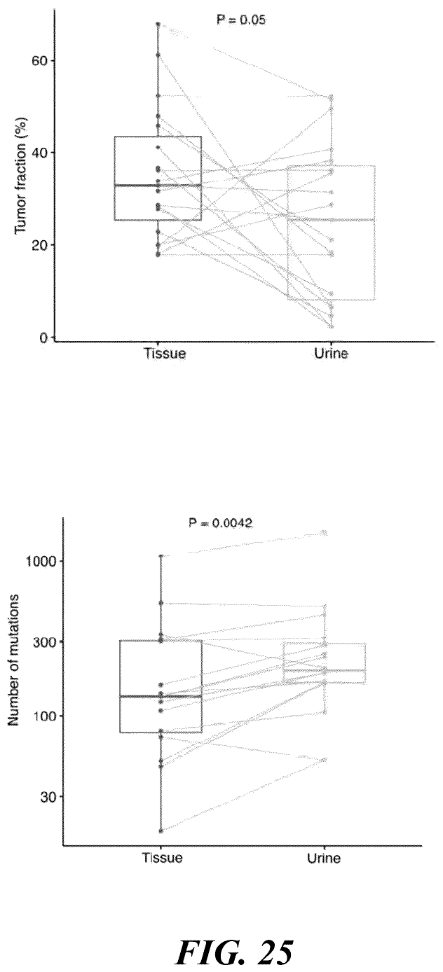

Tumor fractions (TFs) were also inferred from paired urine (2-52%) and tumor tissue (17-68%) showed significant difference (a, p=0.05). FIG. 25 shows plots of tumor fractions in tissue and urine. Though TFs in urine were relatively lower, more somatic mutations were detected in urine than in tumor tissue (b, p<0.05). TMB was also calculated and compared from tDNA and utDNA showing a high correlation (R=0.84).

Overall the results show the effectiveness of urinary tumor DNA as a tissue surrogate for mutation profiling in MIBC at the whole-exome scale, supporting urine-based noninvasive molecular profiling in precision medicine for patients with bladder cancer.

PredicineWES+™, the boosted whole exome sequencing, identified 1493 somatic variants in 11 CSF samples, among which 97 variants were previously reported as possibly pathogenic by public clinical database. For NSCLC-specific biomarkers, 7 out of 11 patients carried EGFR variants including 3 Exon19del incidents, 1 Exon20ins incident, 1 L858R mutation, and other gain-of-function mutations. Additionally, a EML-ALK mutation was detected in one patient.

### Example 7: Monitoring of Breast Cancer Using Blood Samples

Two comprehensive NGS assays were performed to profile somatic mutations and copy number variation in blood samples collected from HR+/HER2-negative metastatic breast cancer patients at baseline and during treatment with endocrine therapy and CIDK4/6 inhibition (ET+CDK4/6i) Specifically, blood samples were evaluated from a phase II study of palbociclib plus letrozole or fulvestrant on a weekly schedule of 5 days on/2 days off, in 28-day cycles, as the first- or second-line treatment.

The first assay used was PredicineWES+, a boosted whole exome sequencing (WES) assay that combines WES with deep coverage of 600 cancer genes targeted by the PredicineATLAS panel, was used to generate exome-wide genomic profiles of somatic single nucleotide variation (SNV), indels and copy number variation (CNV), and to determine blood tumor mutation burden (bTMB) scores reflecting the number of mutations of DNA.

The second assay was PredicineCNB, a low-pass whole genome sequencing (LP-WGS) assay, was used to generate blood copy number burden (bCNB) scores representing a comprehensive measure of copy number variation, including amplifications and deletions across all chromosome arms (e.g., using the PredicineSCORE™ liquid biopsy assay (Predicine, Inc., Hayward, CA)).

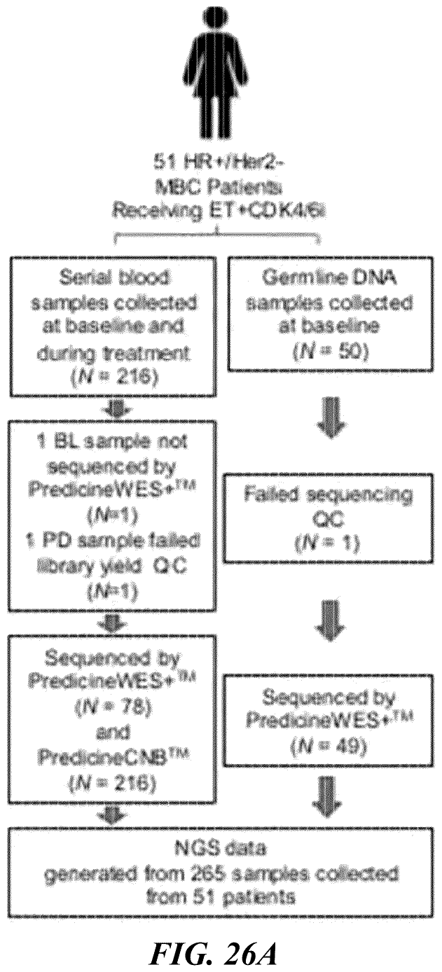

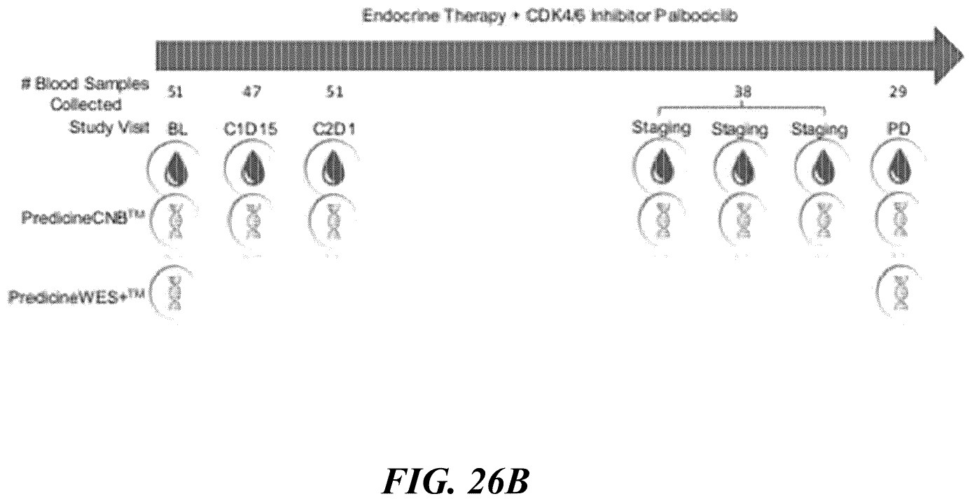

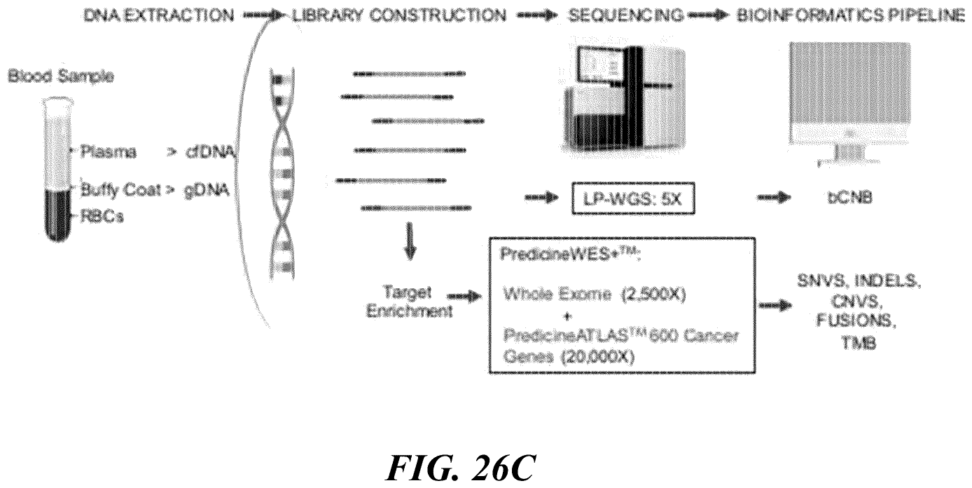

FIGS. 26A-26C show the study schematics, samples collection, and assay timeline and operations. FIG. 26A shows the study schematics. FIG. 26B shows the timeline of the sampling and sequencing. Sample collection at baseline (BL), and during treatment at cycle 1 day 15 (C1D15), cycle 2, day 1 (C2D1), Q3-month staging scans without progressive disease (PD), and at imaging detection of PD. From 51 patients, 216 serial blood samples were taken at baseline and treatment. Additionally, a germline DNA sample was taken from each patient. Following QC operations, 78 blood samples were sequenced using PredicineWES+ and 218 samples were sequenced with PredicineCNB. Similarly 49 of the germline samples were sequenced using PredicineWES+. FIG. 26C shows the NGS profiling with PredicineWES+ and PredicineCNB. A blood sample was separated into a plasma samples comprising cfDNA, buffy coat comprising gDNA, and red blood cells (RBCs). DNA was extracted from the sample, and a library was generated. The library was subjected to two different sequencing workflows. For one part of the sample, low pass whole genome sequencing at a depth of 5× was performed and the reads were analyzed to determine a blood copy number burden. For the second part of the sample, target enrichment was performed to perform a whole exome sequencing (at 2500× depth) with additional enrichment of a specific 600 target genes of the Predicine ATLAS (at 20,000× depth). These reads were then used to identify SNVs, Indels, CNVs, fusion, and determine a tumor mutational burden.

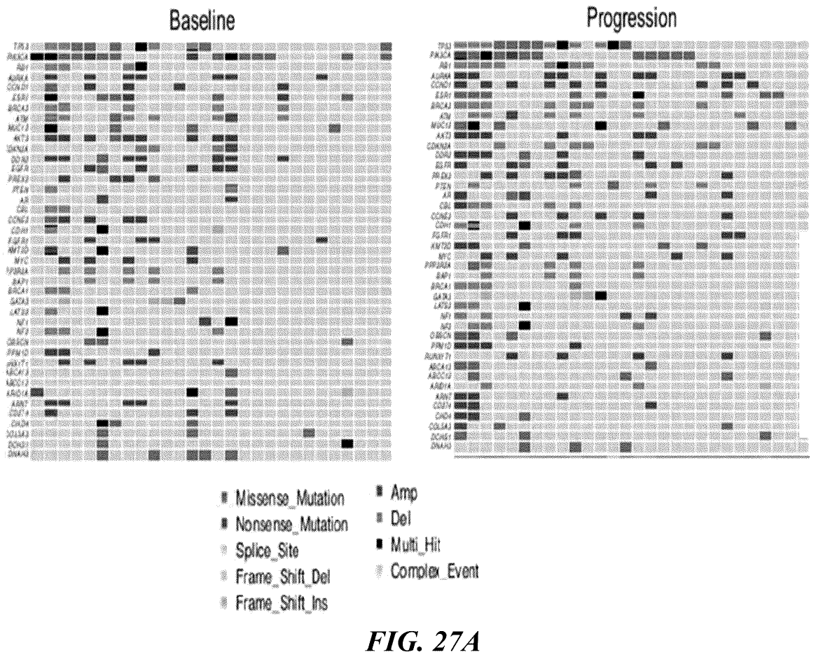

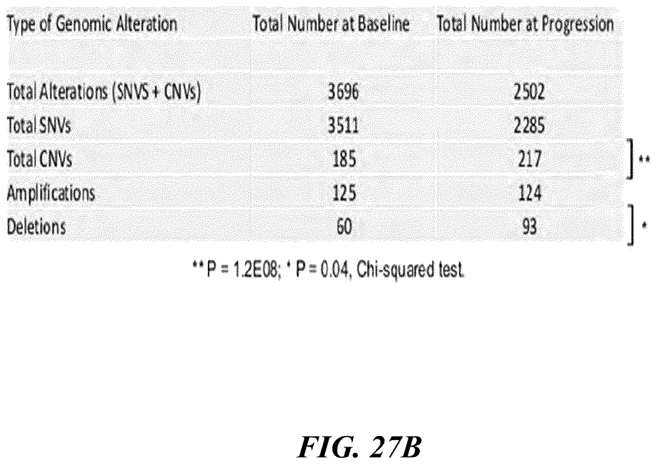

FIGS. 27A-27B show data from the PredicineWES+ assay. FIG. 27A shows a heatmap of the top altered genes in the Baseline and Progression sample. The number and type of genomic alterations as well as the frequency of alteration for specific genes in baseline samples and progression samples were also analyzed. Based on this data, enrichment of specific variants could be identified at progression, and a decrease in total number of SNVs were observed at progression. FIG. 27B shows states relating to the alterations in baseline compared to at progression. However, an increase in the total number of CNVs, primarily copy loss event, were observed in the progression samples. When observing the blood Tumor Mutation Burden for the baseline and progression samples, there was no change in median levels.

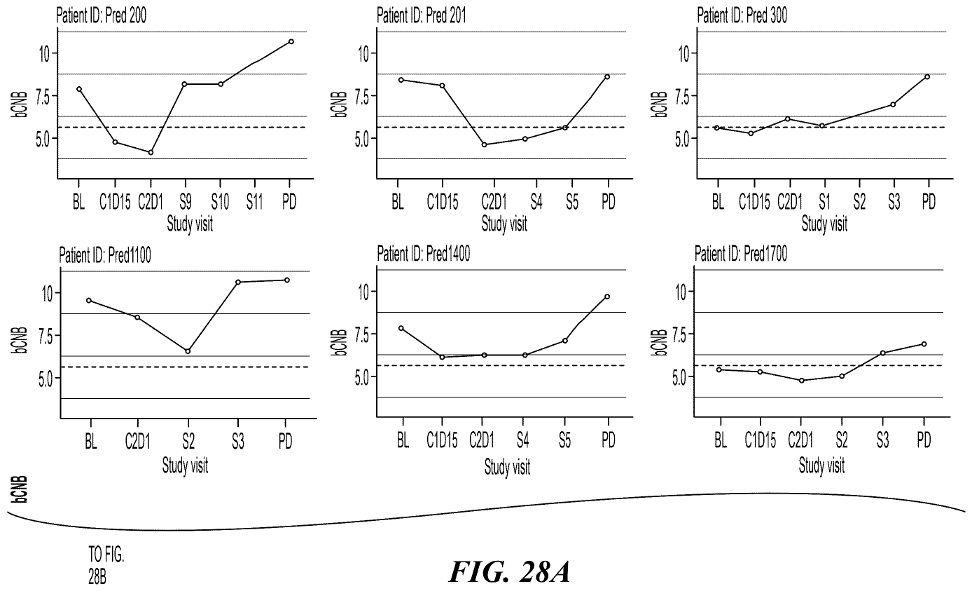

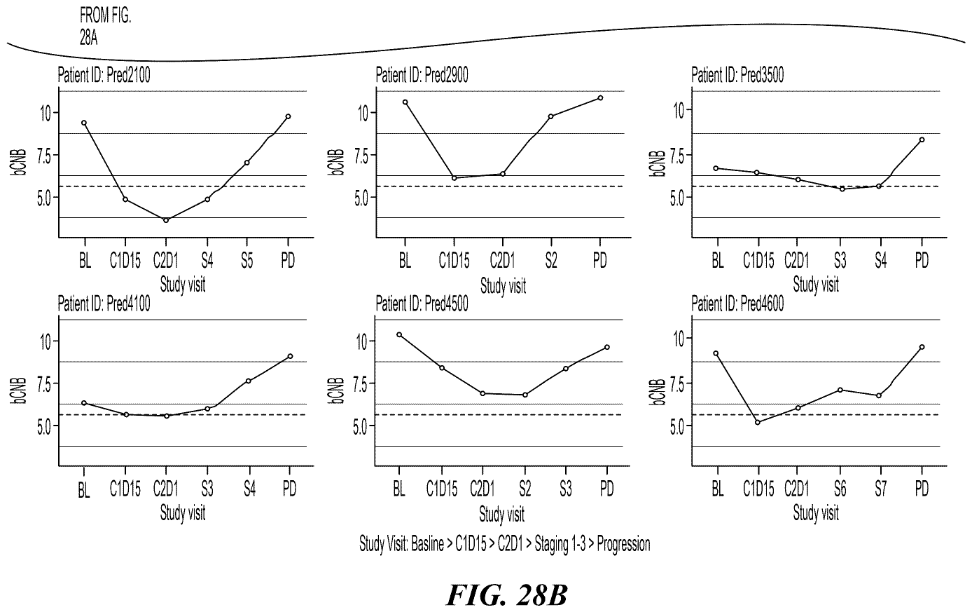

When looking at blood copy number burden (bCNB), the median for the baseline and progression was also similar. However, bCNB was able to track treatment progression. FIG. 28A-28B shows a series of bCNB during treatment and showed decreases at C1D15 and/or C2D1, followed by increases in bCNB that preceded imaging detection of progressive disease in 12/18 (66.7%) of patients for whom staging blood samples were analyzed. As such, bCNB was able to track treatment as well as predict progression prior to any radiographic detection.

As demonstrated in the example, WES in plasma is a highly sensitive, comprehensive NGS approach for detecting individual variants at baseline and during treatment, some of which are significantly enriched at progression. However, NGS assays designed around specific variants to monitor for disease progression are costly. As such, dynamic changes in CNVs during treatment can be detected prior to radiographic detection of relapse using a shallow LP-WGS assay. This approach constitutes a promising cost effective method to serially monitor for early signs of metastatic disease progression during treatment.

### Example 8: Use of ctDNA in Cerebrospinal Fluid for Cancer Detection

Leptomeningeal metastases occur in more than 3% of patients diagnosed with non-small-cell lung cancer (NSCLC) through the whole course of disease, resulting in poor clinical outcomes and limitations on therapies. The cerebrospinal fluid (CSF) is a direct liquid biopsy for pathological diagnosis of leptomeningeal metastases. However, traditional clinical methods of detecting tumor cells in CSF showed limited sensitivity. Meanwhile, the unique genomic aberrations of leptomeningeal metastases remain unknown. Here we report the prospective clinical study aiming to identify genomic aberrations carried by NSCLC patients with leptomeningeal metastases through circulating tumor DNA (ctDNA) in CSF.

In the study, 13 patients were enrolled and CSF samples were collected after diagnosis of metastases. Among them, PBMC samples were collected from 11 patients as germline control materials. PredicineCNB, a lowpass whole-genome sequencing (LP-WGS) assay, was performed to identify copy number variations and tumor fraction in CSF samples from all 13 patients. Furthermore, PredicineWES+, a boosted whole exon sequencing assay, was performed on paired CSF and PBMC samples from 11 patients.

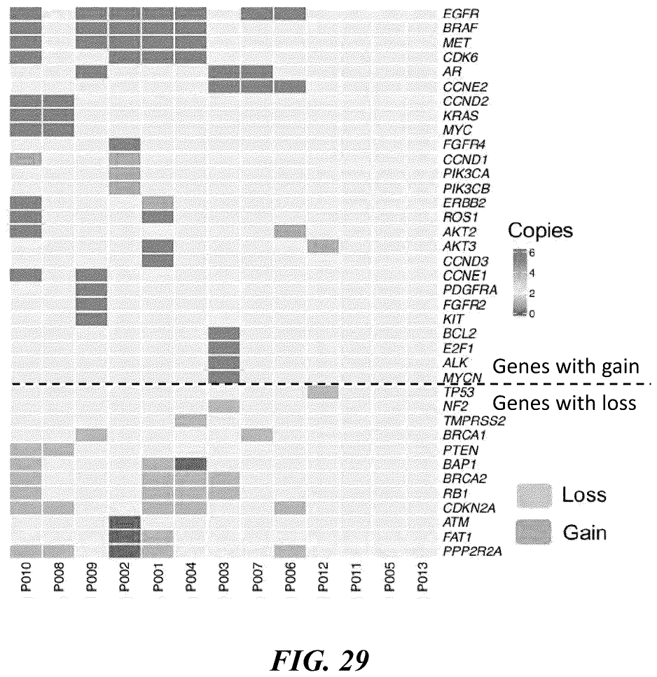

ctDNA fractions were identified in all 13 CSF samples through PredicineCNB™ assay (e.g., using the PredicineSCORE™ liquid biopsy assay (Predicine, Inc., Hayward, CA)). Gene copy variants related to NSCLC were also detected such as copy gain of EGFR(7 pts), BRAF(5 pts), MET(5 pts), KRAS(2 pts), ERBB2(2 pts), ROS1(2 pts), ALK(1 pts) and copy loss of RB1(4 pts), PTEN(2 pts), TP53(1 pts). FIG. 29 shows the copy number gain and loss of determined via PredicineCNB.

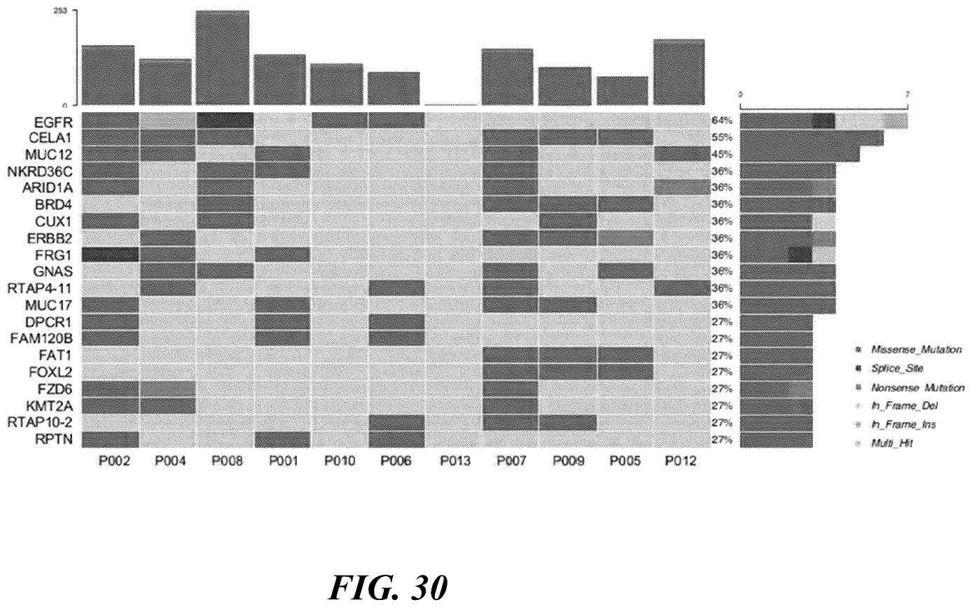

PredicineWES+™, the boosted whole exome sequencing, identified 1493 somatic variants in 11 CSF samples, among which 97 variants were previously reported as possibly pathogenic by public clinical database. For NSCLC-specific biomarkers, 7 out of 11 patients carried EGFR variants including 3 Exon19del incidents, 1Exon20ins incident, 1 L858R mutation, and other gain-of-function mutations. FIG. 30 shows the mutational landscape determined via PredicineWES+.

### Example 9: Analysis of TMB and MSI Using Liquid Biopsy

Tumor mutational burden (TMB) and microsatellite instability (MSI) are emerging biomarkers that correlate with response to immunotherapies, PredicineATLAS is a proprietary NGS-based assay that enables robust measurement of TMB and MSI in cell-free circulating DNA (cfDNA) extracted from blood samples. This report summarizes the analytical validation, including accuracy, specificity, Limit of Detection—minimum tumor content (LOD), and precision (repeatability and reproducibility) of the PredicineATLAS assay.

PredicineATLAS assay uses a 600-gene panel and is designed to measure TMB and MSI in liquid biopsy samples collected from cancer patients. cfDNA was extracted, labeled with unique molecular barcodes during library construction, followed by enrichment using PredicineATLAS panel and then paired-end sequenced using an Illumina platform.

For blood samples, 10 mL of peripheral venous blood was collected in Streck Cell-Free DNA BCT. Upon receipt, samples were processed immediately into plasma and stored at −80° C. cfDNA was extracted by QIAamp circulating nucleic acid kit and quantified by Qubit. Cell line genomic DNA (gDNA) samples were enzyme digested and serially size-selected to mimic the plasma cfDNA profile. Extracted cfDNA was labeled with unique molecular barcodes and the ligated sequencing library was PCR amplified with a high-fidelity polymerase and quantified by Bioanalyzer. For enrichment, sequencing library was blocked with adaptor specific blocking oligonucleotides and hybridized with PredicineATLAS panel. Captured library bound to the beads were amplified and quantified by Bioanalyzer. Enriched libraries were normalized, pooled, and loaded onto the Illumina platform for 2×150 bp paired-end sequencing. Libraries were sequenced to a median depth of >20,000×.

To achieve accurate and robust TMB estimation, only highly confident somatic single nucleotide variant (SNV) mutations in the targeted coding regions were taken into account in the TMB calculation.

NGS data was analyzed using Predicine's DeepSEA NGS analysis pipeline, which starts from the raw sequencing data (BCL files) and outputs the final mutation calls. The pipeline first performs adapter trimming, barcode checking, and error correction. Cleaned paired FASTQ files were aligned to human reference genome build hg19 using BWA alignment tool. Consensus bam files were then derived by merging paired-end reads originated from the same molecules as single strand fragments. Single strand fragments from the same double strand DNA molecules were further merged as double stranded. Both sequencing and PCR errors were corrected during this process.

Following DeepSEA variant caller, variants were filtered based on variant backgrounds from a pool of normal control samples and other historical samples. Other metrics such as base quality, log odds ratio, and distance to fragment ends were used to remove variants with low confidence. A detected call is a variant with at least 4 unique support fragments of which one should be double-stranded.

A germline variant filter can be implemented when matched normal sample is not available. In many applications, liquid biopsy assays may not comprise a matched normal. A germline variant filter can use the assumption that the tumor derived somatic mutations have much lower variant allele frequencies than heterozygous germline variants. This can assume that variants with high allele frequency are germline derived. The variant allele frequency is adjusted by copy number changes when they happen at the variant location. Variants annotated in public germline databases with relatively high population allele frequency are also filtered out.

TMB score was estimated based on the number of somatic SNVs in the coding regions and normalized by the total coding region size covered by the panel. The TMB score was not estimated if the MSAF (maximum somatic allele frequency) is less than the threshold.

MSI score was assessed from a tumor sample by counting the number of unstable markers. For MSI detection, PredicineATLAS assay analyzes 50 MSI markers in the panel, which are short tandem repeat regions in reference genome. The tumor sample is predicted as MSI-High (MSI-H) if the MSI score is greater than a threshold which is defined in the titration experiment (FIG. 6). For each MSI marker, a z-score was calculated by comparing the repeat length distributions of tumor sample and normal background constructed from batch of normal plasma samples. The marker is c The marker is considered unstable if the z-score is above a threshold that is estimated from the validation data. PCR or sequencing noise is suppressed by error correction using DeepSEA algorithm before constructing the repeat length distribution.

PredicineATLAS panel contains 600 cancer-related genes covering 2.4 Mb genomic regions and 1.36 Mb coding regions.

To evaluate PredicineATLAS panel for TMB measurement, TMB scores derived from the assay were compared to TMB scores calculated from the public WES data. 7116 tissue samples spanning >30 tumor types from the public TCGA data downloaded from Broad GDAC Firehose (https://gdac.broadinstitute.org/) were used in the analysis. This in-silico analysis showed that PredicineATLAS panel-based TMB scores highly correlate with WES-based TMB scores (R=0.98, P<0.001).

### Performance Metrics of Predicine NGS Panel

Based on the standard 15 ng DNA input, a satisfactory assay performance of calling base substitutions, indels, re-arrangements, copy number gain (CNG), and copy number loss (CNL) is summarized in Table 14.

TABLE 14
Summary of Performance metrics

| | Reportable | AF/Copy | | Sensitivity | PPV |
|---|---|---|---|---|---|
| Variant type | Ranges | Number | | (%) | (%) |
| SNVs | ≥0.05% | <0.25% | AF | 78.3 | 97.9 |
| | | 0.25-0.5% | AF | 98.6 | 99.2 |
| | | ≥0.5% | AF | 100 | 100 |
| Indels | ≥0.05% | <0.25% | AF | 80 | 100 |
| | | 0.25-0.5% | AF | 98.6 | 100 |
| | | ≥0.5% | AF | 100 | 100 |
| DNA | ≥0.05% | <0.25% | AF | 33.3 | 100 |
| re-arrangements | | 0.25-0.375% | AF | 90 | 100 |
| | | 0.375-0.5% | AF | 96.7 | 100 |
| | | ≥0.5% | AF | 100 | 100 |
| Copy number | ≥2.18 | ≥2.375 | copies | 100 | 100 |
| gain | copies | 2.23-2.375 | copies | 100 | 100 |
| | | <2.23 | copies | 45.0 | 81.8 |
| Copy number | ≤1.85 | 1.80-1.85 | copies | 66.0 | 88.6 |
| loss | copies | 1.75-1.80 | copies | 93.6 | 91.7 |
| | | ≤1.75 | copies | 100 | 100 |

Eight cell lines with WES data available from COSMIC database (https://cancer.sanger.ac.uk/cosmic/) were tested by PredicineATLAS assay for TMB measurement. TMB scores from PredicineATLAS assay were demonstrated to be highly correlated with the TMBs from the public WES data (, R=0.97, P<0.001).

### LoD of the Assay

For TMB, the LoD is defined as the lowest tumor content required to obtain at least 90% concordance between detected TMB status and expected TMB status. Four cell lines with different TMBs were titrated into five different levels of tumor contents (20%, 10%, 1%, 0.5% and 0.25%), and each level has a minimum of two replicates. The resulting dilution samples have tumor content ranging from 0.25% to 20%. All samples were fragmented and size selected to mimic the size of plasma cfDNA. As these cancer cell lines carry copy number changes across the whole genome, the MAFs (mutation allele frequency) range from 10% to 90%. To facilitate the TMB LoD evaluation, only cell line mutations with MAF>35% and no CNV regions were included in the TMB LoD evaluation. High correlation of TMB scores between PredicineATLAS panel and WES data from COSMIC was observed.

To evaluate the LoD, the concordance between detected and expected TMB status was evaluated for different TMB cut-offs (5, 10, 15, 20, 30 Muts/Mb) respectively. The assay reached at least 90% concordance with LoD 1%.

For MSI, the LoD is defined as the lowest titration level at which at least 90% MSI-High samples can be detected as MSI-H samples. To determine the LoD for MSI, MSI-H cell lines was serially diluted in a MSS cell line targeting multiple titration levels (20%, 10%, 1%, 0.5%, 0.25%). Samples with >1% tumor cells, 100% were detected as MSI-H and 90.9% samples at 1% titration level were identified as MSI-H. Therefore, the MSI assay reached 90.9% concordance with as low as 1% tumor content.

Accuracy was established by comparing the TMB scores calculated from a serial of titrated cell lines with expected TMB scores at TMB cut-off 10 Muts/Mb. In total, 39 samples with ≥21% tumor content were used for the TMB accuracy. All samples with high TMB (TMB score≥10) are categorized as high and all samples with low TMB (TMB score<10) are categorized as low. None of the samples was mis-categorized, therefore, the PPV of the PredicineATLAS TMB assay is established as 100%.

For MSI, accuracy was established by comparing the MSI status calculated from a serial of titrated cell lines with expected MSI status. In total, 21 MSI samples with 21% tumor content and 9 MSS samples were used for the MSI accuracy. The accuracy of the MSI is established as 96.7% (95% CI: 82.8-99.9%).

To assess the performance of the PredicineATLAS assay and ensure that a “blank” sample does not generate analytical signals, TMB and MSI scores of cfDNA from healthy donors were evaluated using the PredicineATLAS assay for specificity. All the samples from the healthy donors have 0 TMB score (with AF cut-off threshold=0.5%) and are predicted as MSS (FIG. 6). 100% (95% CI: [69.2-100%]) of the samples are categorized as TMB low with cutoff 10 Muts/Mb and MSS with 14 cutoff for MSI. These baseline data suggest the PredicineATLAS assay has 100% specificity for the TMB and MSI measurement.

### Repeatability (Intra-Assay Precision)

To evaluate the closeness of agreement between repeated tests of the same samples under the same operating conditions, 8 replicate groups with at least two replicates per group were performed under the same operating conditions for TMB and MSI analysis respectively. High similarity was observed for all the replicate samples.

To evaluate the closeness of agreement between the results of measurement when operating conditions are varied, reproducibility was assessed and compared across different reagent lots, operators and sequencers. A set of 6 samples with either high or low TMBs were performed at different conditions and TMB scores were compared between replicates. A set of 8 samples with MSI-H status were performed at different conditions and MSI status were compared between replicates. High concordance was observed in all the replicate samples for both TMB and MSI studies. Cell line at 1% titration level has been processed for multiple times across seven-month period using the PredicineATLAS assay. The measured TMB scores (sorted by processing time) are shown in FIG. 7. The difference between measured and expected TMB score (157.36) is ranged from −5.6% to 6.5%, indicating a high reproducibility of the PredicineATLAS assay.

PredicineATLAS analyzes 600 cancer-related genes in a single assay to provide assessment of immunotherapy biomarkers (TMB and MSI). The assay underwent rigorous analytical validation testing using 15 ng of low cfDNA input amount. The validation study demonstrated that PredicineATLAS has high concordance with WES for accurate assessment of TMB. Furthermore, evaluation of MSI status showed high concordance with cell line samples. The development of the cfDNA-based PredicineATLAS assay provides a complementary approach to the tissue-based TMB and MSI assay for patients with solid tumors. PredicineATLAS is a robust and high-performance assay that allows a concurrent measurement of TMB and MSI in liquid biopsy samples.

### Example 10: Detection of Blood Cancers

Blood cancers mainly start in the bone marrow and account for about 10% of all the newly diagnosed cancer patients. As an alternative to invasive bone marrow biopsies that are currently used to monitor blood cancers, comprehensive genomic profiling of cell-free circulating DNA (cfDNA) or peripheral blood mononuclear cell (PBMC) in blood provides a minimally invasive and clinical convenient solution for detecting genomic biomarkers to guide clinical decisions in oncology.

PredicineHEME™ is a capture-based targeted next-generation sequencing (NGS) assay that enables accurate detection of genomic alterations, including small nucleotide variants (SNVs), insertions and deletions (indels), copy number variants (CNVs) and DNA re-arrangements, in plasma cfDNA or genomic DNA (gDNA) extracted from PBMC or bone marrow aspirate (BMA) for patients with hematologic malignancies.

PredicineHEME™ is unique in its ability to detect variant allele frequency down to 0.1% in plasma cfDNA or blood/BMA gDNA. This example presents a summary of the analytical validation of the 106-gene PredicineHEME™ assay, including accuracy, specificity, sensitivity, and precision. PredicineHEME™ analyzes 106 key blood cancer-relevant genes for potential genomic biomarkers in blood cancer patients. The capture-based NGS assay is designed to detect SNVs, indels, copy number variations, and gene re-arrangements in plasma, blood or BMA samples collected from patients with blood cancers.

Extracted plasma cfDNA or the enzymatically fragmented gDNA are labeled with unique molecular barcodes during library construction, followed by enrichment using the PredicineHEME™ panel and then paired-end sequenced using Illumina platform. Peripheral venous blood is collected in Streck Cell-Free DNA BCT if plasma isolation is needed, otherwise EDTA tube is used for whole blood sample collection. Upon receipt, samples are accessioned and processed immediately into plasma or buffy coat and stored at −80° C.

Plasma cfDNA is extracted by QIAamp circulating nucleic acid kit, and PBMC or BMA genomic DNA (gDNA). samples are extracted using DNeasy Blood & Tissue Kit. Genomic DNAs are further enzyme digested and purified before DNA quantification and quantitation. Extracted cfDNA or fragmented gDNA is labeled with unique molecular barcodes and the ligated sequencing library is PCR amplified with a high-fidelity polymerase and quantified by Bioanalyzer. For enrichment, sequencing library is blocked with adaptor specific blocking oligonucleotides and hybridized with the PredicineHEME™ panel. Captured library bound to the beads is amplified and quantified by Bioanalyzer. Enriched libraries are normalized, pooled and loaded onto the Illumina platform for 2×150 bp paired-end sequencing. Libraries are sequenced to a median depth of >20,000×.

Coupled with in-house developed DeepSEA variant calling software for high accuracy in variant detection, PredicineHEME™ assesses SNVs, indels, re-arrangements, and CNVs in one workflow. NGS data is analyzed using Predicine's proprietary DeepSEA NGS analysis pipeline, which starts from the raw sequencing data (BCL files) and outputs the final mutation calls. The pipeline performs adapter trimming, barcode checking, and error correction. Cleaned paired FASTQ files are then aligned to human reference genome build hg19 using BWA alignment tool. Consensus BAM files are then derived by merging paired-end reads originated from the same molecules as single strand fragments. Single strand fragments from the same double strand DNA molecules are further merged as double stranded. Both sequencing and PCR errors are corrected during this process.

Following DeepSEA variant caller, variants are filtered based on variant backgrounds from a pool of normal control and other historical samples. Other metrics such as base quality, log odds ratio, entropy and distance to fragment ends are used to remove variants with low confidence.

A germline variant filter can be implemented when matched normal sample is not available. The variant allele frequency is adjusted by copy number changes when it happens at the variant location. It assumes adjusted variants with allele frequency near 50% or above 90% are germline derived. Variants annotated in public germline databases (e.g. 1000 Genomes Project) with relative high population allele frequency are also filtered out.

SNV and Indel Variants are called after filtering low base quality and low mapping score reads. Detected variants are further filtered based on variant background (defined by normal plasma samples and historical data), repeat regions and other quality metrics. Benign and likely benign SNPs are excluded in the variant call list. Copy number variation (CNV) is estimated at the gene level. The pipeline first calculates the on-target unique fragment coverage based on consensus bam files then adjusts the GC-bias coverage. The unique fragment coverage profiles from a group of normal samples are used as references to normalize and estimate the z-score of the copy number changes. DNA re-arrangement is detected by identifying the alignment breaking points based on the bam files before the consensus operation. Suspicious alignments are filtered based on repeat regions, local entropy calculation and similarity between reference and alternative alignments. Larger or equal to 2 unique alignments are used to report a DNA fusion.

To evaluate the accuracy of the assay, reference samples with known genomic alterations in a diversity of the assayed gene were tested, which included cell lines, commercial reference samples and patient samples tested by a 3rd party laboratory. Up to 30 ng cfDNA or fragmented gDNA input was analyzed by the PredicineHEME™ assay.

Concordance was determined by comparing the expected variants from the reference samples with the detected variants by the assay and used as performance indicators to demonstrate accuracy. In total, 102 mutations from 41 samples were expected to be detected by this panel assay and all the mutations were confirmed with 100% PPA [96.6-100%]. In this validation study, the Limit of Detection (LoD) is defined as the lowest mutant allele frequency at which a variant type could be reliably detected in 95% of replicates by the PredicineHEME™ assay. Studies were conducted to demonstrate a putative sensitivity value for each variant type including SNVs, indels, DNA re-arrangements, and CNGs. Reference cfDNA samples with defined MAFs at 4 different levels (0.1, 0.25, 0.375, 0.5%) were evaluated to define the LOD for each of the variant type. Both PPA and PPV for each mutation level were calculated and used for defining the LoD.

For SNV, a total of 80 expected variants were evaluated with an expected 0.25% AF, and the assay achieved 98.75% sensitivity (95% CI, 93.2-100%) with 97.53% PPV (95% CI, 91.4-99.7%). For Indel, a total of 20 expected variants were evaluated with an expected 0.375% AF, and the assay achieved 100% sensitivity (95% CI, 83.2-100%) with 100% PPV (95% CI, 83.2-100%). For DNA re-arrangement, a total of 20 expected variants were evaluated with an expected 0.375% AF, and the assay achieved 95% sensitivity (95% CI, 75.1-99.9%) with 100% PPV (95% CI, 82.4-100%). For CNG, a total of 20 expected variants were evaluated with expected 2.23 copies, and the assay achieved 100% sensitivity (95% CI, 83.2-100%) with 100% PPV (95% CI, 83.2-100%). Based on the analytical validation data, the LoD of SNV is 0.25% MAF, the LoD of indel is 0.375% MAF, the LoD of DNA re-arrangement is 0.375% MAF, and the LoD of CNG is 2.23 copies for MYC gene.

To evaluate the specificity of the assay and ensure that a “blank” sample does not generate analytical signals, 24 samples including buffy coat, plasma and BMA from healthy donors were tested. Analytic specificity was estimated based on the number of false positive mutations within the targeted panels. Analytic Specificity=100*(1−# of false positives/Panel size).

Although health donors may also have low variant frequency pathogenic mutations, such as CHIP mutations. We consider all variants passing detection criteria of the NGS analysis pipeline as false positives including CHIP mutations. The analytic specificity is 99.9999%.

Precision was measured by the variation of estimated variant frequency between replicates. All samples in the precision study were assessed for the entire workflow, from the library to sequencing analysis. In total, 18 sets of the same sample aliquots were used for the precision study and each set contains reference standard with defined MAF above the LoD and NTC (no template control).

Repeatability test includes intra-run performance (samples processed under the same conditions). Three replicates of each sample were performed at the same condition and results were compared. Reproducibility was assessed based on samples independently processed on 5 days by at least 2 operators. 100% concordance between replicates were detected for both intra-run and inter-run precision studies.

102 clinical gDNA samples from patients with hematologic indications were analyzed with the PredicineHEME™ assay. In total, 714 genomic alterations, including SNVs, indels, copy number variations were detected in 98 out of 102 total samples. Copy number variations were detected in 13 genes.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-117. (canceled)

118. A method for detecting a presence or an absence of cancer in a subject, comprising:
(a) assaying cell-free deoxyribonucleic acid (cfDNA) molecules from a biological sample obtained or derived from said subject, wherein the biological sample comprises a urine sample;
(b) detecting a set of biomarkers from said cfDNA molecules wherein said set of biomarkers comprise differentially expressed markers or variants;
(c) computer processing said set of biomarkers to detect said presence or said absence of said cancer in said subject.

119. The method of claim 118, wherein at least one of said cfDNA molecules are assayed using nucleic acid sequencing to produce nucleic acid sequencing reads.

120. The method of claim 119, wherein said nucleic acid sequencing is selected from the group consisting of: next-generation sequencing, whole genome sequencing, low-pass sequencing, targeted sequencing, whole exome sequencing, methylation-aware sequencing, bisulfite sequencing, and a combination thereof.

121. The method of claim 119, wherein said nucleic acid sequencing comprises targeted sequencing.

122. The method of claim 118, wherein said cancer is selected from the group consisting of: breast cancer, lung cancer, prostate cancer, colorectal cancer, melanoma, bladder cancer, non-Hodgkin lymphoma, kidney cancer, endometrial cancer, leukemia, pancreatic cancer, thyroid cancer, and liver cancer, and any combination thereof.

123. The method of claim 118, further comprising identifying a clinical intervention for said subject based at least in part on said detected presence or said absence of said cancer.

124. The method of claim 123, wherein said clinical intervention is selected from the group consisting of: surgical resection, chemotherapy, radiotherapy, immunotherapy, adjuvant therapy, neoadjuvant therapy, androgen deprivation therapy, and a combination thereof.

125. The method of claim 123, further comprising administering said clinical intervention to said subject.

126. The method of any of claim 118, wherein said set of biomarkers comprises quantitative measures of a set of cancer-associated genomic loci.

127. The method of claim 126, wherein said set of cancer-associated genomic loci comprises one or more members selected from the group consisting of genes listed in Table 1 or Table 2.

128. The method of claim 126, wherein said set of cancer-associated genomic loci comprises PTEN, TP53, RB1, FGFR3, or ERBB2.

129. The method of any one of claim 126, further comprising using probes or primers configured to selectively enrich said biological sample for nucleic acid molecules corresponding to a set of genomic loci.

130. The method of any of claim 118, further comprising monitoring said presence or said absence of said cancer in said subject, wherein said monitoring comprises assessing said presence or said absence of said cancer in said subject at each of a plurality of time points.

131. The method of any of claim 118, wherein said set of biomarkers from said cfDNA molecules comprise tumor-associated alterations selected from the group consisting of: copy number alterations (CNAs), copy number losses (CNLs), single nucleotide variants (SNVs), insertions or deletions (indels), and rearrangements.

132. The method of claim 118, further comprising determining, among said set of biomarkers, a mutant allele frequency of a set of somatic mutations.

133. The method of claim 118, further comprising determining a blood copy number burden based on copy number alterations or copy number losses of said set of biomarkers.

134. The method of claim 132, further comprising determining a circulating tumor DNA (ctDNA) fraction of said cancer of said subject based at least in part on said set of mutant allele frequencies.

135. The method of any of claim 132, further comprising determining a tumor mutational burden (TMB) of said cancer of said subject based at least in part on said set of mutant allele frequencies.

136. The method of claim 132, further comprising determining an abnormality score of said cancer of said subject based at least in part on said set of mutant allele frequencies.

137. A method for detecting a presence or an absence of cancer in a subject, comprising:
(a) providing cell-free deoxyribonucleic acid (cfDNA) molecules from a biological sample obtained or derived from said subject;
(b) performing a first sequencing assay on said cfDNA, or derivatives thereof, to generate a copy number data of at least one region of a genome of a subject, wherein the sequencing is performed at a depth of no more than 10×;
(c) performing a second sequencing assay on said cfDNA molecules or derivatives thereof, wherein the second sequencing assay comprises a whole exome sequencing assay or a methylation-aware sequencing assay to generate sequencing data;
(d) computer processing said copy number data and said sequencing data to detect a set of biomarkers; and
(e) based at least on the presence of said set of biomarkers, detecting said presence or said absence of said cancer in said subject.
